(19) United States
(12) Patent Application Publication
(10) Pub. No.: US 2008/0015465 A1
(43) Pub. Date: Jan. 17, 2008

(54) METHODS FOR DIAGNOSING AND TREATING PAIN IN THE SPINAL CORD

(76) Inventor: Gaetano J. Scuderi, Jupiter, FL (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/763,412

(22) Filed: Jun. 14, 2007

Related U.S. Application Data
(60) Provisional application No. 60/813,848, filed on Jun. 15, 2006.
(60) Provisional application No. 60/883,840, filed on Jan. 8, 2007.

(51) Int. Cl. A61B 10/00

(52) U.S. Cl. 600/563; 604/503

(57) ABSTRACT

The present invention provides methods, reagents and kits for the diagnosis and treatment of spinal-related pain, e.g., radiculopathic pain, facet pain and discogenic pain. Cytokine biomarkers, e.g., IFNγ can be used to diagnose spine-related injury. It is also a finding of the present invention that spinal-related pain can be alleviated by administering therapeutic agents to the site of diagnostic presence of the cytokine biomarker. This invention further provides methods for extraction of samples from the spine, e.g., disc space samples, epidural samples and facet joint samples.

### CROSS-REFERENCES TO RELATED APPLICATIONS

This application claims benefit of U.S. provisional application No. 60/813,848, filed Jun. 15, 2006; and U.S. provisional application No. 60/883,840, filed Jan. 8, 2007, each of which are herein incorporated by reference in their entirety for all purposes.

### BACKGROUND OF THE INVENTION

The manifestation of radiculopathic pain has traditionally been attributed to various physical/mechanical abnormalities, such as compression or mechanical irritation of the nerve root related to conditions such as disk herniation, stenosis, spondylolisthesis, Piriformis Syndrome, Obturator Syndrome, various types of cysts, e.g. ganglion and synovial, tumors, and the like.

It has recently been demonstrated that application of nucleus pulposus to the spinal nerve root can result in axonal damage and functional changes to nerve root micro-anatomy, resulting in pain-related behaviors. Thus, it has been theorized that a mechanical defect releasing the nucleus pulposus into the epidural space may cause nerve root damage resulting in radicular pain. This “Chemical Radiculopathy” along with theories regarding auto-immune responses to the release of nucleus pulposus, have been advanced to explain the manifestation of radiculopathic pain in only a portion of a patient population that has mechanical failures such as disk herniation, while the remaining patients remain pain-free.

Various pro-inflammatory cytokines have been implicated in the pathogenesis of back pain. For example, tumor necrosis factor-alpha (TNF-α) has been shown to cause irritation to nerve roots, and is alleged to be involved in inflammation related to leakage of nucleus pulposus. Additionally, high levels of interleukin-1 (IL-1) were found in periadicular and extraneural tissues in patients with a herniated nucleus pulposus compared to controls (see, e.g. Cooper et al. Spine 20:591-598 1995). Increased levels of interleukin-1-beta, macrophages and other inflammatory cells were also observed in disk tissue of patients with a herniated nucleus pulposus versus control patients, although the results were variable (see, e.g. Gronblade et al., Spine 19:2744-2751 1994). In another study, tissue adjacent to nerve roots at the site of disk herniation was harvested during surgery for analysis of cytokine and inflammatory cell content. Interleukin-1-beta (IL-1-beta), interleukin-1-alpha (IL-1-alpha), interleukin-6 (IL-6), tumor necrosis factor-alpha (TNF-alpha), granulocyte-macrophage colony stimulating factor (GM-CSF) and prostaglandin E2 (PGE2) were detected. The cytokines were produced primarily by histiocytes, fibroblasts and endothelial cells. However, the levels of these cytokines were determined in comparison with various types of disk herniation, but not to control values (Takahashi et al. Spine 21:218-224 1996). In tissue culture, disks that were herniated spontaneously produced higher levels of matrix metalloproteinases, nitric oxide, IL-6, and PGE2. Interestingly, TNF-alpha, IL-1-beta, and IL-1-alpha were not found in herniated or control disks (Kang et al. Spine 21:271-277 1996).

In additional studies, PGE2 was higher in certain types of disk herniations, and tended to be correlated with clinical findings (straight leg test) (O'Donnell et al. Spine 21(14):1653-1655; 1996).

Inflammatory cells (macrophages, T-cells), fibroblasts and endothelial cells expressing some chemokines (monocyte chemotactic protein 1 and macrophage inflammatory protein-1-alpha) were found to be higher in a group of patients having herniated nucleus pulposus than in a control group (Haro et al. Spine 21914):1647-1652 1996).

Interleukin-12 (IL-12) and interferon-gamma (INF-γ) were found in higher concentrations in human herniated nucleus pulposus disks rather than in contained disks. In contrast, the concentration of IL-4 was higher in contained disks compared to non-contained disks (Park et al. Spine 27(19):2125-2128 2002).

Prostaglandins and leukotrienes are known to cause sensitization of nociceptors and thus play a role in the pathogenesis of pain. Saal et al. (Spine 15:674-678 1990) identified a high level of phospholipase A2, which leads to the production of prostaglandins and leukotrienes, in symptomatic disk herniations, which may lead to radicular pain. The presence of prostaglandin-like substances in radicular disk pain may explain the beneficial effect of corticosteroids in the management of disk herniation symptoms, as corticosteroids inhibit the activation of phospholipase A2 and block the production of prostaglandins and leukotrienes.

Recently, other cytokines have been identified that may play a role in the pathophysiology of disk-related pain syndromes. Several studies have identified tumor necrosis factor alpha (TNF-α) and interleukin-8 (IL-8) in surgical disk specimens that were removed for the treatment of pain. TNF-alpha has been shown to influence local blood flow and vascular permeability as well as accelerate the inflammatory response. (Brisby et al. European Spine Journal 11:62-66 2002; Olmarker et al. Spine 26(8):863-9 2001; Cannon et al. Mol Cell Biochem 179:159-167 1998). Burke et al. (Journal of Bone and Joint Surgery-British volume 84(2):196-201 2002) identified high levels of both interleukin-6 (IL-6) and interleukin-8 (IL-8) in patients with symptomatic degenerative disk disease who underwent fusion. It has been postulated that even a small amount of these factors may be sufficient to initiate an inflammatory process after rupture of the nucleus pulposus due to their ability to recruit other cytokine-producing cells and stimulate up-regulation of genes encoding pro-inflammatory mediators (Koes et al. Pain Digest 9:241-247 1999).

Several studies have suggested a possible role for pain-related neuropeptides in symptomatic intervertebral disk disease (McCarron et al. Spine 12:760-764 1987). Substance P (SP) and calcitonin gene-related peptide (CGRP) have been evaluated in the cerebrospinal fluid (CSF) and dorsal root ganglion of human and animal models respectively, and have been demonstrated to be elevated by local disk inflammation (Ohtori et al. Autonomic Neuroscience-Basic & Clinical 86(1-2):13 2000; Ohtori et al. Annals of Anatomy 184(3):235-240 2002; Lindh et al. Neuropeptides 33(6):517-521 1999; Lindh et al. Scand. Journal of Rheumatology 26(6):468-472 1997). However, the site of production of these molecules and the specific role they play in patient symptoms remains to be determined.

Studies by Olmarker, Cuckler et al., and Koes et al., which examined biopsy specimens during clinical removal of disk material in the treatment of lumbar herniated nucleus pulposus have claimed that that anti-inflammatory mediators are most likely involved in the majority of sciatic-like symptoms secondary to lumbar disk herniation (Olmarker, Schmerz., December 2001; 15(6): 425-9; Boden et al., JBJS 1990; 72A:403-8; Cuckler et al., JBJS. 1985; 67A:63-66; Koes et al., Pain Digest. 1999; 9:241-7).

Tobinick (U.S. Pat. No. 6,982,089) discloses methods for treating neurological or neuropsychiatric diseases or disorders in humans by administering to humans a therapeutically-effective dose of specific biologics. The biologics considered by Tobinick are antagonists of tumor necrosis factor (TNF) or of interleukin-1 (IL-1). Olmarker (U.S. Pat. No. 7,115,557) discloses to use of TNFα inhibitors for the treatment of nerve root injury.

Chappell et al (US Pre-grant Publication No. 20060094056 B1) provide methods for diagnosing, treating, or evaluating inflammatory and autoimmune disease by sampling bodily fluids from a human subject having a suspected diagnosis. The bodily fluid samples are analyzed for the presence and amount of certain cytokines, which provides the diagnosis, prognosis or evaluation of therapeutic response.

Despite the myriad of studies designed to elucidate the apparent role of inflammatory mediators in the pathophysiology of spinal-related pain, relatively little has been discovered concerning the molecular aspects of the associated pain and the specific biochemical molecules involved. In many of the aforementioned studies the results have been inconclusive and contradictory at best.

Once the inflammatory mediators are elucidated and directly linked to the symptoms, these mediators will provide new targets for the development of diagnostics and therapeutic tools. Although some studies have provided evidence that the epidural compartment may be affected by an intervertebral disk herniation, none have measured concentrations of biomolecules in the epidural space in an attempt to detect the differences between affected and non-affected persons. It is important that the causes of the structural changes that accompany disk herniation and the mediators responsible for the incitement of discomfort be identified and thus allow for targeted and effective therapy. This invention addresses this and other problems relating to radiculopathy.

### BRIEF SUMMARY OF THE INVENTION

The invention is based, in part, on the discovery that spinal-related pain, e.g., radiculopathic pain or discogenic pain can be diagnosed based on the presence of biomarkers in samples from the spine. In some instances, interferon-gamma (IFNγ) or a fragment of IFNγ is present in the epidural space and/or disk space of patients with spinal-related pain and one or more mechanical abnormalities associated with degenerative disk disease or neuro-compressive disease. Thus, in one aspect the invention provides a method of diagnosing a patient that is a candidate for certain therapies, where the method comprises detecting the presence of a biomarker, e.g., IFNγ or a fragment of IFNγ in a sample from the spine.

In some embodiments, the present invention provides a method of identifying a cytokine biomarker of spinal-related pain, the method comprising: a) providing a biological sample from the spine of a patient suspected to be suffering from radiculopathic pain, and b) detecting the cytokine biomarker in the biological sample from the spine. In some embodiments, the cytokine biomarker is Interferon gamma (IFN-γ) or a fragment of IFN-γ or protein which reacts with IFN-γ antibody.

In some embodiments, the biological sample for the present invention is a sample from the spine obtained using a technique selected from the group consisting of epidural space lavage, transforaminal epidural space lavage, translaminar epidural space lavage, epidural caudal sponge retrieval, transforaminal caudal sponge retrieval, disk space lavage, disk space caudal sponge retrieval or facet lavage or aspiration.

In some embodiments, the cytokine biomarker of the present invention is identified by an immunoassay. In some embodiments, the cytokine biomarker of the present invention is identified based on the presence of the nucleic acid encoding the cytokine biomarker. In some embodiments, detection of the nucleic acid is by an amplification reaction. In some embodiments, the amplification reaction is a polymerase chain reaction.

In some aspects, the present invention provides a method of selecting a patient for treatment, wherein the patient is suspected of having radiculopathic pain or discogenic pain or facetogenic pain, the method comprising: detecting the level of IFNγ or a fragment of IFNγ in a biological sample from the spine, wherein the presence of IFNγ or a fragment of IFNγ is indicative of a patient to be selected for treatment.

In some embodiments, the biological sample is a sample from the spine obtained using a technique selected from the group consisting of epidural space lavage, transforaminal epidural space lavage, translaminar epidural space lavage, epidural caudal sponge retrieval, transforaminal caudal sponge retrieval, disk space lavage, disk space caudal sponge retrieval or facet lavage or aspiration.

In some embodiments, detecting the presence of IFNγ or a fragment of IFNγ comprises an immunoassay. In other embodiments, detecting the level of IFNγ or a fragment of IFNγ comprises detecting the level of nucleic acid. In some embodiments, detecting the presence of the nucleic acid is by an amplification reaction, typically a polymerase chain reaction.

In some embodiments, the method further comprises administering one or more therapeutic agents to the patient, wherein the one or more therapeutic agents are selected from the group consisting of an INFγ antagonist and a steroidal anti-inflammatory agent. In some embodiments, the method comprises administering an IFNγ antagonist to the patient in an amount sufficient to reduce the level of pain. In some embodiments, the IFNγ antagonist is an anti-IFNγ antibody. In some embodiments, the antibody is a neutralizing antibody. In some embodiments, the antibody is a monoclonal antibody. In some embodiments, the monoclonal antibody is a humanized antibody.

In some embodiments, the method of the present invention further comprises administering at least one additional therapeutic agent. In some embodiments, the additional therapeutic agent is an anti-inflammatory agent. In some embodiments, the anti-inflammatory agent is a steroidal anti-inflammatory agent.

In some embodiments, the present invention provides a kit for diagnosing radiculopathic pain, the kit comprising: a) an antibody panel comprising an antibody to IFNγ, and b) a device for extraction of a biological sample from the spine. In some embodiments, the kit comprises the device for biological sample extraction from the spine directly connected to a chamber comprising the antibody panel.

### DETAILED DESCRIPTION OF THE INVENTION

I. Introduction

This invention relates generally to methods for identifying one or more cytokine biomarkers associated with radiculopathic pain, discogenic pain in a patient having one or more mechanical abnormalities associated with degenerative disk disease or neurocompressive disease. In particular, the invention relates to a method for identification of a cytokine biomarker, e.g. interferon-gamma (interferon-γ), or a fragment thereof, or a cytokine or chemokine that is immunoreactive with an anti-IFNγ antibody, e.g., anti-IFNγ antibody available from Sanquin (Sanquin, Amsterdam, Netherlands), in a sample from the epidural space or disk space and/or facet joint of patients manifesting spinal related pain and/or radiculopathy. In some embodiments, the cytokine biomarker or a fragment of the cytokine biomarker indicative of radiculopathy or discogenic disease in a patient is detected in a spine sample from the patient using the anti-IFNγ antibody present in the Bio-Rad 17-plex panel available from Bio-Rad under catalog number 171A11171 (Bio-Rad, Hercules, Calif.).

The present invention provides methods, reagents, and kits for diagnosing spinal-related pain, e.g., radiculopathy, facet pain or discogenic pain. The invention is partly based upon the discovery that a cytokine biomarker can be used to diagnose and differentiate spinal related injury, e.g., Radiculopathy, discogenic or facet injury. In some embodiments, the presence of the cytokine biomarker of the present invention is indicative of injury at a particular location in the spine where the cytokine biomarker is detected. In some embodiments a protein or peptide biomarker from a spine sample immunoreactive with an anti-IFNγ antibody is indicative of a spinal related injury in the patient. In some embodiments a protein or peptide biomarker from a spine sample that is immunoreactive with an anti-IFNγ antibody is indicative of discogenic or radiculopathic pain in the patient. In some embodiments, a patient suffering from spinal-related pain can be diagnosed with radiculopathy, discogenic or facet injury based on the presence of a polypeptide or protein biomarker that is immunoreactive with the anti-INFγ antibody in the Bio-Rad 17-plex panel available under catalog number 171A11171 (Bio-Rad, Hercules, Calif.).

The ability to diagnose radiculopathy, discogenic or facet pain by detecting a cytokine biomarker, e.g., IFNγ or a fragment of IFNγ, provides a method of identifying or diagnosing a patient that is a likely candidate for therapy, e.g., anti-inflammatory therapeutic agents, including those that block IFNγ activity. The detection of the cytokine biomarker, e.g., IFNγ, or a fragment of IFNγ can also be used to monitor the efficacy of a treatment for spinal pain. For example, the level of or a cytokine biomarker that reacts with the anti-INFγ antibody in the Bio-Rad 17-plex panel available under catalog number 171A11171 (Bio-Rad, Hercules, Calif.) can be assessed after treatment and compared to the level before the treatment. A decrease in the level of the cytokine biomarker after the treatment indicates efficacious treatment. Similarly, the nucleotide level of the cytokine biomarker, e.g., IFNγ can be assessed in a spine sample from a patient experiencing pain and the nucleotide levels of the biomarker can be indicative of a patient in need of treatment. The presence or level of the nucleotide coding for a cytokine biomarker can additionally be used to assess efficacy of treatment.

In some embodiments, assessing the presence or level of a cytokine biomarker, e.g., IFNγ or a fragment thereof, in a spine sample from a patient experiencing spinal-related pain can be used to determine whether the patient is candidate for treatment. In some embodiments the presence or level of IFNγ in a spine sample can be used to determine what type of treatment is most appropriate for the patient, e.g., steroidal therapy, IFNγ blockers, surgery etc. In some embodiments, the presence or level of a cytokine biomarker, e.g., a cytokine biomarker reactive with an anti-IFNγ antibody, in the epidural space is indicative of a patient who is likely to benefit from a therapeutic agent, e.g., IFNγ antagonist injected into the epidural space. In some embodiments, the presence or level of a cytokine biomarker, e.g., a cytokine biomarker reactive with an anti-IFNγ antibody, in the disk space is indicative of a patient who is likely to benefit from a therapeutic agent, e.g., IFNγ antagonist injected into the disc space. In some embodiments, the presence or level of a cytokine biomarker, e.g., a cytokine biomarker reactive with an anti-IFNγ antibody in the facet joint is indicative of a patient who is likely to benefit from a therapeutic agent, e.g., IFNγ antagonist injected into the facet joint.

In some embodiments, the present invention provides a method to localize spinal-related injury when such injury is not diagnosable by other presently available methods, e.g., MRI. In some embodiments the presence or level of the cytokine biomarker of the present invention at a particular location in the spine is indicative of injury at that particular location and is used to select a patient who can benefit from a therapeutic agent at that particular location, e.g., in the case of a patient experiencing acute pain. In some embodiments, detection of the cytokine biomarker anywhere in the spine is indicative of chronic pain. In some embodiments, patients suffering from chronic spinal pain are selected for systemic administration of a therapeutic agent.

In some embodiments, the present invention provides methods for sample extraction from the spine, e.g., from the epidural space or from the disk space or from the facet joint. In some embodiments, the present invention provides methods for the administration of therapeutics into the spine, e.g., directly at the site where the cytokine biomarker is detected. In some embodiments, the method of sample extraction and therapeutic administration are performed concurrently. In some embodiments, the same device used for sample extraction is adapted for therapeutic administration.

II. Definitions

“Radicular pain,” “radiculopathy,” “radiculopathic pain” and “sciatica” refer to radiating pain of the extremities which emanates from the spinal root level or “radic” along the path of one or more irritated lumbar nerve roots. In the case of sciatica, this would originate from the L4, L5 and/or S1 spinal nerve roots, which make up the sciatic nerve. Radiating pain is also possible from the high lumbar disk herniations in the L3, L2 or L1 regions or from any cervical nerve root in the case of a cervical disk herniation, cervical nerve root irritation or cervical disk degeneration This pain differs from pain resulting from a facet joint or other spinal structure, which is classified as “referred” pain. Radiating pain is also possible from the high lumbar disc herniations in the L3, L2 or L1 regions or cervical spine regions.

“Discogenic pain” as used herein refers to spinal-related pain that generates from the disk. The intervertebral disk suffers from reduced mechanical functionality secondary to a loss of hydration from the nucleus pulposus. The reduction in the ability of the disc to transmit loads evenly and efficiently through the vertebral bodies leads to damage in the outer region known as the annulus fibrosus. This weakening can lead to fissuring, tears or “chemical” leaking of the disk that may manifest in a herniation or possibly lead to spinal related pain, including radiculopathy.

“facet joint pain” as used herein refers to pain generating from the facet joint. “Facet joints” or “zygapophysial joints” are paired, true synovial joints endowed with cartilage, capsule, meniscoid, and synovial membrane.

The term “biomarker” as used herein refers to any protein or polypeptide fragment or full length cytokine or chemokine which diagnostic presence in a spine sample from a patient suffering or suspected to be suffering from radiculopathic pain, discogenic pain or facet pain can be differentiated from a spine sample from a normal or control (asymptomatic) patient. The presence or level of the “cytokine biomarker” can conveniently be detected using anti-INFγ antibody in the Bio-Rad 17-plex panel available under catalog number 171A11171 (Bio-Rad, Hercules, Calif.). The “cytokine biomarker” of the present invention can be interferon gamma or a fragment thereof In some embodiments, the cytokine biomarker of the present invention is further defined by its inability to be detected using the following ELISA kits as per the manufactures' written protocols: BD Biosciences Pharmigen's Human IFN-gamma ELISA Kit, OptEIA, catalog number 550612 (BD Biosciences, San Jose, Calif.), the R&D Systems' Human IFN-gamma Quantikine ELISA Kit, catalog number DIF50 (R&D Systems, Minneapolis, Minn.), the eBioscience's Human IFNγ ELISA Kit, Ready-SET-Go!, catalog number 88-7916-29 (eBioscience, San Diego, Calif.).

The term “IFNγ,” “interferon gamma” or “gamma interferon” as used herein refers to an interferon gamma protein or peptide or any protein or peptide or cytokine biomarker, that is immunoreactive with and can be detected using an anti-INFγ antibody or multiple anti-INFγ antibodies included in the Bio-Rad 17-plex panel available under catalog number 171A11171 (Bio-Rad, Hercules, Calif.). An exemplary human interferon gamma amino acid sequence can be found under the NCBI accession number AAB59534. “IFNγ” as used herein also includes any naturally-occurring “IFNγ” variant, e.g., a splice variant or mutant, e.g., associated with a disease or a disorder. The term “IFNγ fragment” or “fragment of IFNγ” refers to any fragment of IFNγ of 20, 30, 40, 50 or more amino acids from IFNγ that can be detected with an anti-interferon gamma antibody, e.g., an interferon gamma antibody included in the Bio-Rad 17-plex panel. The IFNγ fragment can also be a fragment of a homolog, mutant or post-translationally modified variant of IFNγ. The presence or level of IFNγ can conveniently be detected using the anti-INFγ antibodies included in the Bio-Rad 17-plex panel available under catalog number 171A11171 (Bio-Rad, Hercules, Calif.), used either as a capture and/or detection antibody within the Bio-Rad 17-plex panel or as the capture and/or detection antibody within a separate assay such as ELISA or other immunoassay).

“Spine sample” or “sample from the spine” as used herein is a sample of tissue or fluid from the spine including “spinal disk sample,” “epidural sample” and “facet joint sample.” Frequently, these samples are also referred to as “biological sample.” As used herein, “biological sample” refers to a cell or population of cells or a quantity of tissue or fluid from a patient. Such samples are typically from humans, but include tissues isolated from non-human primates, rodents, e.g., mice, and rats, caprines, bovines, canines, equines and felines. Biological samples may also include sections of tissues such as biopsy samples, frozen sections taken for histologic purposes, and lavage samples.

The “level of a cytokine biomarker” or the “level of IFNγ protein or polypeptide” or “the level of a fragment of IFNγ” in a biological sample refers to the amount of polypeptide that is present in a cell or biological sample. The polypeptide may or may not have cytokine activity, e.g., IFNγ protein activity. A “level of cytokine biomarker” or “level of IFNγ protein or its fragment” need not be quantified, but can simply be detected, e.g., a subjective, visual detection by a human, with or without comparison to a level from a control sample or a level expected of a control sample.

The “level of IFNγ mRNA” in a biological sample refers to the amount of mRNA encoding IFNγ that is present in a cell or a biological sample. The mRNA generally encodes a kind of IFNγ protein. A “level of IFNγ mRNA” need not be quantified, but can simply be detected, e.g., a subjective, visual detection by a human, with or without comparison to a level from a control sample or a level expected of a control sample.

The terms “polypeptide,” “peptide” and “protein” are used interchangeably herein to refer to a polymer of amino acid residues. The terms apply to amino acid polymers in which one or more amino acid residues is an artificial chemical mimetic of a corresponding naturally occurring amino acid, as well as to naturally occurring amino acid polymers, those containing modified residues, and non-naturally occurring amino acid polymers.

The term “acute pain” as used herein refers to pain lasting up to six months, e.g., five months, four months, three months, two months, four weeks, three weeks, two weeks, one week, six days, five days, four days, three days, two days or one day or less.

The term “chronic pain” as used herein refers to pain of a duration of longer than six months.

The term “antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. The singular term “an antibody” as used herein is understood to encompass plural referents unless the context clearly indicates otherwise. In some instances the plurality of the antibodies can belong to the same antibody species, e.g., in the case of monoclonal antibodies, while in some cases different antibodies species are encompassed the by phrase “an antibody,” e.g., a polyclonal antibodies.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g. McCafferty et al., Nature 348:552-554 (1990)). When referring to treatment methods, antibodies that are humanized or otherwise specific to the species to be treated are used.

For preparation of antibodies, e.g., recombinant, monoclonal, or polyclonal antibodies, many technique known in the art can be used (see, e.g. Kohler & Milstein, Nature 256:495-497 (1975); Kozbor et al., Immunology Today 4:72 (1983); Cole et al., pp. 77-96 in Monoclonal Antibodies and Cancer Therapy (1985); Coligan, Current Protocols in Immunology (1991); Harlow & Lane, Antibodies, A Laboratory Manual (1988); and Goding, Monoclonal Antibodies: Principles and Practice (2d ed. 1986)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The term “agent” or “therapeutic agent” is used to describe a compound that has or may have a pharmacological activity. Agents include compounds that are known drugs, compounds for which pharmacological activity has been identified but that are undergoing further therapeutic evaluation, and compounds that are members of collections and libraries that are to be screened for a pharmacological activity. The term includes an organic or inorganic chemical such a peptide, including antibodies, proteins and small molecules and natural products.

The term “immunoassay” refers to an assay that uses an antibody or antibodies to specifically bind an antigen. The immunoassay is characterized by the use of specific binding properties of a particular antibody or antibodies to isolate, target, and/or quantify the antigen.

“Specific binding” between a binding agent, e.g., an antibody and a protein, for instance, a biomarker cytokine, refers to the ability of a capture- or detection-agent to preferentially bind to a particular cytokine that is present in a mixture; e.g., a fluid from a joint. Specific binding also means a dissociation constant (K_{D}) that is less than about 10⁻⁶ M; preferably, less than about 10⁻⁸ M; and, most preferably, less than about 10⁻⁹ M.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample.

A “label” or a “detectable moiety” is a composition detectable by spectroscopic, photochemical, biochemical, radiographic, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into the peptide or used to detect antibodies specifically reactive with the peptide. The labels may be incorporated into nucleic acids, proteins and antibodies at any position. Any method known in the art for conjugating the antibody to the label may be employed, e.g., using methods described in Hermanson, Bioconjugate Techniques 1996, Academic Press, Inc., San Diego.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe. Alternatively, methods using high affinity interactions may achieve the same results where one of a pair of binding partners binds to the other, e.g., biotin, streptavidin.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not functionally interfere with hybridization. Thus, e.g. probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence. Diagnosis or prognosis may be based at the genomic level, or at the level of RNA or protein expression.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

The phrase “functional effects” in the context of assays for testing compounds that modulate activity of a cytokine biomarker, e.g., IFNγ protein includes the determination of a parameter that is indirectly or directly under the influence of the cytokine biomarker, e.g., IFNγ protein or nucleic acid, e.g., a functional, physical, or chemical effect, such as, e.g., the ability to decrease inflammation.

By “determining the functional effect” is meant assaying for a compound that increases or decreases a parameter that is indirectly or directly under the influence of a cytokine biomarker, e.g., IFNγ protein sequence, e.g., functional, enzymatic, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, radiographic or solubility properties for the protein, measuring inducible markers or transcriptional activation of the cytokine biomarker protein; measuring binding activity or binding assays, e.g. binding to antibodies or other ligands, and detecting inflammation. Determination of the functional effect of a compound can be evaluated by many means known to those skilled in the art, e.g., microscopy for quantitative or qualitative measures of alterations in morphological features, measurement of changes in RNA or protein levels for the cytokine biomarker sequences, e.g., IFNγ sequences, measurement of RNA stability, identification of downstream or reporter gene expression (CAT, luciferase, β-gal, GFP and the like), e.g., via chemiluminescence, radiography, fluorescence, colorimetric reactions, antibody binding, inducible markers, and ligand binding assays.

“Inhibitors” as used herein directly or indirectly partially or totally block activity, decrease, prevent, delay activation, inactivate, desensitize, or down regulate the activity or expression of the cytokine biomarker. “Antagonists” as used herein directly, e.g., binding o the cytokine biomarker, reduce the level or activity of the cytokine biomarker. Antagonists are, for example, polypeptides, such as antibodies, soluble receptors and the like, as well as nucleic acids such as siRNA or antisense RNA, genetically modified versions of the cytokine biomaker, e.g., versions with altered activity, as well as naturally occurring and synthetic cytokine biomarker antagonists, small chemical molecules and the like. Assays for detecting inhibitors include, e.g. expressing the cytokine biomarker protein, e.g., IFNγ in vitro, in cells, or cell membranes, applying putative antagonist compounds, and then determining the functional effects on the cytokine biomarker activity, e.g., IFNγ activity, as described above.

III. Identification of Cytokine Biomarker Sequences in a Sample from a Patient

In one aspect of the invention, the expression levels of a cytokine biomarker, e.g. IFNγ or a fragment of IFNγ, are determined in different patient samples, e.g., a lavasate from the spine for which diagnostic or prognostic information is desired. That is, radiculopathic, discogenic and facet pain may be diagnosed and/or distinguished from other types of spinal pain, e.g., muscular pain or pain frequently associated with persons suffering from depression. In some embodiments, the cytokine biomarker is identified or its level is assessed based on reactivity with an anti-IFNγ antibody, for example the anti-INFγ antibody in the Bio-Rad 17-plex panel available under catalog number 171A11171 (Bio-Rad, Hercules, Calif.).

In some embodiments, the presence or level of the cytokine biomarker, e.g., IFNγ or a fragment of IFNγ can be used to select a patient as candidate for treatment. In some other embodiments, the presence or level of the cytokine biomarker, e.g. IFNγ or a fragment of IFNγ can be used to determine the success during the course of or after treatment of spinal related pain, e.g., radiculopathic pain, discogenic pain or facet pain.

A. Methods for Detecting Cytokine Biomarkers

Immunoassays can be used to qualitatively or quantitatively analyze a cytokine biomarker, e.g., the levels of IFNγ or a fragment of IFNγ in a biological sample. A general overview of the applicable technology can be found in a number of readily available manuals, e.g., Harlow & Lane, Cold Spring Harbor Laboratory Press, Using Antibodies: A Laboratory Manual (1999).

In addition to using immunoassays to detect cytokine biomarkers in a sample from the spine, assessment of cytokine biomarker expression and levels can be made based at the level of gene expression. RNA hybridization techniques for determining the presence and/or level of mRNA expression are well known to those of skill in the art and can be used to assess the presence or level of gene expression of the cytokine biomarkers of interest.
a) Antibodies and Immunoassays

In some embodiments, the methods and kits of the present invention utilize selective binding partners of a cytokine biomarker, e.g., IFNγ or its fragment to identify its presence or determine its levels in a sample from the spine. The selective binding partner to be used with the methods and kits of the present invention can be, for instance, an antibody. In some aspects, monoclonal antibodies to the cytokine biomarker can be used. In some other aspects, polyclonal antibodies to the cytokine biomarker can be employed to practice the methods and in the kits of the present invention.

Commercial antibodies to a variety of cytokine biomarkers, for instance, commercial antibodies to IFNγ are readily available and can be used with the methods and kits of the present invention. It is well know to those of skill in the art that the type, source and other aspects of an antibody to be used is a consideration to be made in light of the assay in which the antibody is used. In some instances, antibodies that will recognize its antigen target (for instance, an epitope or multiple epitopes from IFNγ) on a Western blot might not be applicable to all ELISA or ELISpot assay and vice versa. In some embodiments the following anti-IFN-gamma antibody can be used to practice the methods of the present invention: the anti-INFγ antibody in the Bio-Rad 17-plex panel available under catalog number 171A11171 (Bio-Rad, Hercules, Calif.).

In some embodiments, the antibodies to be used for the assays of the present invention can be produced using techniques for producing monoclonal or polyclonal antibodies that are well known in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g. Huse et al., Science 246:1275-1281 (1989); Ward et al., Nature 341:544-546 (1989)). Such antibodies can be used for therapeutic and diagnostic applications, e.g., in the treatment and/or detection of IFNγ-associated diseases or conditions described herein.

By way of example, when using IFNγ as a cytokine biomarker of the present invention, a number of immunogens from IFNγ may be used to produce antibodies specifically reactive with IFNγ and fragments of IFNγ. For example, a recombinant IFNγ or an antigenic fragment thereof, can be isolated using methods well known to those of skill in the art. Recombinant protein can be expressed in eukaryotic or prokaryotic cells. Recombinant protein is the typically used immunogen for the production of monoclonal or polyclonal antibodies. Alternatively, a synthetic peptide derived from the known sequences of IFNγ and conjugated to a carrier protein can be used as an immunogen. Naturally-occurring protein may also be used either in pure or impure form. The product is then injected into an animal capable of producing antibodies. Either monoclonal or polyclonal antibodies may be generated, for subsequent use in immunoassays to measure the protein.

Once specific antibodies to the cytokine biomarker or cytokine biomarkers of interest are available, each specific cytokine biomarker can be detected by a variety of immunoassay methods. For a review of immunological and immunoassay procedures, see Basic and Clinical Immunology (Stites & Terr eds., 7th ed. 1991). Moreover, the immunoassays of the present invention can be performed in any of several configurations, which are reviewed extensively in Enzyme Immunoassay (Maggio, ed., 1980); and Harlow & Lane, supra.

Immunological binding assays (or immunoassays) typically use an antibody that specifically binds to a protein or antigen of choice (in this case, a cytokine biomarker, e.g., IFNγ or antigenic subsequence or fragment thereof). As described above, the antibody may be produced by any of a number of means well known to those of skill in the art and as described above.

Specific binding of a cytokine biomarker, e.g., IFNγ to an antibody may typically require an antibody that is selected for its specificity. For example, polyclonal antibodies raised to IFNγ can be selected to obtain only those polyclonal antibodies that are specifically immunoreactive with IFNγ, and not with other proteins, except for polymorphic variants, orthologs, and alleles of IFNγ. This selection may be achieved by subtracting out antibodies which react with the cytokine of interest. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically the signal of a specific or selective reaction will be at least twice background signal or noise and more typically more than 10 to 100 times background. Antibodies that react only with a particular IFNγ ortholog, e.g., from specific species such as rat, mouse, or human, can also be detected as described above, by subtracting out antibodies that bind to IFNγ from another species.

Typically, polyclonal antisera with a titer of 10⁴ or greater are selected and tested for their cross reactivity against non-IFNγ proteins or even other related proteins from other organisms, using a competitive binding immunoassay. Specific polyclonal antisera and monoclonal antibodies will usually bind with a K_{d} of at least about 0.1 mM, more usually at least about 1 μM, optionally at least about 0.1 μM or better, and optionally 0.01 μM or better. Similar techniques and principles can be applied when determining reactivity and binding specificity of other antibody/cytokine biomarker combinations of the present invention.

Immunoassays also often use a labeling agent to specifically bind to and allow for the detection of the complex formed by the antibody and antigen. The labeling agent may itself be one of the moieties comprising the antibody/antigen complex. Thus, the labeling agent may be a labeled antibody to a particular cytokine biomarker, e.g., an anti-IFNγ antibody. Alternatively, the labeling agent may be a third moiety, such a secondary antibody, that specifically binds to the antibody/antigen complex (a secondary antibody is typically specific to antibodies of the species from which the first antibody is derived). Other proteins capable of specifically binding immunoglobulin constant regions, such as protein A or protein G may also be used as the label agent. These proteins exhibit a strong affinity for immunoglobulin constant regions from a variety of species (see, e.g. Kronval et al., J. Immunol. 111: 1401-1406 (1973); Akerstrom et al., J. Immunol. 135:2589-2542 (1985)). The labeling agent can be modified with a detectable moiety, such as biotin, to which another molecule can specifically bind, such as streptavidin. A variety of detectable moieties are well-known to those skilled in the art.

Throughout the assays, incubation and/or washing steps may be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C. In some embodiments, the immunological assay is instantaneous and a read-out for the presence or levels of the cytokine biomarkers is available nearly immediately upon extracting the sample from the acutely painful joint and performing the immunoassay.

Immunoassays for detecting cytokine biomarkers, e.g., IFNγ or a fragment thereof, in samples may be either competitive or noncompetitive. Noncompetitive immunoassays are assays in which the amount of antigen is directly measured. In one preferred “sandwich” assay, for example, the antibody to a particular cytokine biomarker, e.g., anti-IFNγ antibodies can be bound directly to a solid substrate on which they are immobilized. These immobilized antibodies then capture the cytokine biomarker protein or fragments thereof, e.g., IFNγ protein or fragments thereof present in the test sample. The cytokine biomarker is thus immobilized and then bound by a labeling agent, such as a second antibody bearing a label. Alternatively, the second antibody may lack a label, but it may, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

In competitive assays, the amount of the cytokine biomarker, e.g., IFNγ or IFNγ fragment(s) present in the sample, e.g., a lavasate from the spine, is measured indirectly by measuring the amount of a known, added (exogenous) cytokine biomarker, e.g., IFNγ displaced (competed away) from an anti-IFNγ antibody by the unknown cytokine biomarker, e.g., IFNγ present in a sample. In one competitive assay, a known amount of a particular cytokine biomarker, e.g., IFNγ is added to a sample and the sample is then contacted with an antibody that specifically binds to that particular cytokine biomarker, e.g., IFNγ. The amount of exogenous cytokine biomarker, e.g., IFNγ bound to the antibody is inversely proportional to the concentration of IFNγ or fragment of IFNγ present in the sample. In one embodiment, the antibody is immobilized on a solid substrate. The amount of cytokine biomarker bound to the antibody may be determined either by measuring the amount of cytokine biomarker present in a cytokine-biomarker/antibody complex, or alternatively by measuring the amount of remaining uncomplexed protein. The amount of the cytokine biomarker may be detected by providing a labeled cytokine biomarker molecule, e.g., a labeled IFNγ molecule.

A hapten inhibition assay is another competitive assay. In this assay the cytokine biomarker, e.g., IFNγ is, for example, immobilized on a solid substrate. A known amount of anti-cytokine biomarker antibody, e.g., anti-IFNγ antibody, is added to the sample, and the sample is then contacted with the immobilized cytokine biomarker, e.g., IFNγ. In the example in which IFNγ is the cytokine biomarker of interest, the amount of anti-IFNγ antibody bound to the known immobilized IFNγ is inversely proportional to the amount of IFNγ present in the sample. As in the embodiment described above, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev. 5:34-41 (1986)).

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred. In addition to, or in place of proteinaceous material, various detergents can be incorporated into the immunoassay to minimize non-specific interactions.

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most labels useful in such methods can be applied to the present invention. Thus, a label is any composition detectable by spectroscopic, photochemical, biochemical, immunochemical, radiographic, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound. The ligands and their targets can be used in any suitable combination with antibodies that recognize the cytokine biomarkers, or secondary antibodies that recognize the antibodies to the cytokine biomakers.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

Detection methods employing immunoassays are particularly suitable for practice at the point of patient care. Such methods allow for immediate diagnosis and/or prognostic evaluation of the patient. Point of care diagnostic systems are described, e.g., in U.S. Pat. No. 6,267,722 which is incorporated herein by reference. Other immunoassay formats are also available such that an evaluation of the biological sample can be performed without having to send the sample to a laboratory for evaluation. Typically these assays are formatted as solid assays where a reagent, e.g., an antibody is used to detect the cytokine. Exemplary test devices suitable for use with immunoassays such as assays of the present invention are described, for example, in U.S. Pat. Nos. 7,189,522; 6,818,455 and 6,656,745. In some embodiments of the present invention an anti-cytokine biomarker antibody, for example, an anti-IFNγ antibody (such as for instance the anti-INFγ antibody in the Bio-Rad 17-plex panel available under catalog number 171A11171 (Bio-Rad, Hercules, Calif.)) can be incorporated into the detection device and allow for cytokine biomarker detection at the point of care, e.g., physician's office.
b) Detection of Polynucleotides

In some aspects, the present invention provides methods for detection of polynucleotide sequences which code for a cytokine biomarker or a fragment thereof, e.g., IFNγ or a fragment of IFNγ in a biological sample, e.g., a spine lavasate for the diagnosis of radiculopathic, facet or discogenic pain. As noted above, a “biological sample” refers to a cell or population of cells or a quantity of tissue or fluid from a patient. Most often, the sample has been removed from a patient, but the term “biological sample” can also refer to cells or tissue analyzed in vivo, i.e., without removal from the patient. Typically, a “biological sample” will contain cells from the patient, but the term can also refer to non-cellular biological material, such as non-cellular fractions of the fluid from a potentially affected disk space, facet joint or epidural space.

Amplification-Based Assays

In one embodiment, amplification-based assays are used to measure the presence or level of a cytokine biomarker, e.g., IFNγ or a fragment thereof In such an assay, the nucleic acid sequences act as a template in an amplification reaction (e.g. Polymerase Chain Reaction, or PCR). In a quantitative amplification, the amount of amplification product will be proportional to the amount of template in the original sample. Comparison to appropriate controls provides a measure of the copy number of the cytokine biomarker. Methods of quantitative amplification are well known to those of skill in the art. Detailed protocols for quantitative PCR are provided, e.g. in Innis et al. (1990) PCR Protocols, A Guide to Methods and Applications, Academic Press, Inc. N.Y.). RT-PCR methods are well known to those of skill (see, e.g., Ausubel et al., supra). In some embodiments, quantitative RT-PCR, e.g., a TaqMan® assay, is used, thereby allowing the comparison of the level of mRNA in a sample with a control sample or value. The known nucleic acid sequences for a particular cytokine biomarker, e.g., IFNγ are sufficient to enable one of skill to routinely select primers to amplify any portion of the gene. Such an exemplary nucleic acid sequence for human IFNγ, can be found, for instance, under accession number NM_{—}000619. Suitable primers for amplification of specific sequences can be designed using principles well known in the art (see, e.g. Dieffenfach & Dveksler, PCR Primer: A Laboratory Manual (1995)).

In some embodiments, a TaqMan® based assay is used to quantify the cytokine biomarker-associated polynucleotides. TaqMan® based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, e.g., AmpliTaq®, results in the cleavage of the TaqMan® probe. This cleavage separates the 5′ fluorescent dye and the 3′ quenching agent, thereby resulting in an increase in fluorescence as a function of amplification (see, for example, literature provided by Perkin-Elmer, e.g., www2.perkin-elmer.com).

In some embodiments, hybridization-based assays can be used to detect the amount of the cytokine biomarker in the cells of a biological sample. Such assays include dot blot analysis of RNA as well as other assays, e.g., fluorescent in situ hybridization, which is performed on samples that comprise cells. Other hybridization assays are readily available in the art.

IV. Sample Retrieval Methods

Any number of methods known to those of skill in the art can be used to retrieve sample from the spine and used with the methods of the present invention. This invention also provides methods for retrieval of biological samples. Examples of such retrieval methods include:

Method 1: Epidural Space Lavage (Caudal)
1) the skin and subcutaneous tissue is infiltrated in the area of introduction that has been verified fluoroscopically;
2) an introducer needle is then inserted through the sacral hiatus, again fluoroscopic confirmation is noted;
3) the catheter is then passed through the needle into the epidural space, utilizing fluoroscopy the catheter is passed to the level of pathology;
4) upon achieving satisfactory position of the catheter the guide wire is removed;
5) a syringe containing approximately 3 cc normal saline (NS) is attached to the distal end of the catheter;
6) ½ cc increments of NS are then injected into the epidural space with an attempt at aspiration with each volume of injectate; three to five seconds is allowed to elapse following each injection prior to re-aspiration;
7) the aspirate is then placed in a microcentrifuge tube containing a protease inhibitor cocktail solution and immediately placed on ice or dry ice or submerged in liquid nitrogen or any other method of rapid freezing;
8) the sample is then transferred to permanent storage at −20° C. or lower until analysis.

Method 2: Epidural (Caudal Sponge)
1) the skin and subcutaneous tissue is infiltrated in the area of introduction that has been verified fluoroscopically;
2) an introducer needle is then inserted through the sacral hiatus, again fluoroscopic confirmation is noted;
3) the catheter is then passed through the needle into the epidural space, utilizing fluoroscopy the catheter is passed to the superior most aspect of the level of pathology;
4) the introducer guide wire is removed and the analyte wire with absorbent material is introduced;
5) a syringe containing 1 cc of normal saline (NS) is attached to the introducer port;
6) NS is then injected soaking the cotton sponge;
7) the catheter is then drawn back to the inferior most aspect of the pathology with the cotton sponge being drawn over the lesion;
8) the analyte wire is then removed;
9) the composition is then washed into a microcentrifuge tube containing a protease inhibitor cocktail solution and immediately placed on ice or dry ice or submerged in liquid nitrogen or any other method of rapid freezing;
10) the sample is then transferred to permanent storage at −20° C. or lower until analysis. Alternatively, at step nine the composition could then be immediately analyzed utilizing a centralized lab analysis or point of care assay.

Method 3: Transforaminal (Epidural Modification)
1) the skin and subcutaneous tissue is infiltrated in the area of introduction that has been verified fluoroscopically;
2) a small bore needle is then inserted through into the affected foramen, again fluoroscopic confirmation is noted;
3) a syringe containing approximately 1.5 cc normal saline (NS) is attached to the needle;
4) approximately ½ cc increments of NS are then injected into the epidural space with an attempt at aspiration with each volume of injectate, three to five seconds is allowed to elapse following each injection prior to reaspiration;
5) the aspirate is then placed in a microcentrifuge tube containing a protease inhibitor cocktail solution and immediately placed on ice or dry ice or submerged in liquid nitrogen or any other method of rapid freezing;
6) the sample is then transferred to permanent storage at −20° C. or lower until analysis.

Method 4: Transforaminal (Absorbent Sponge)
1) the skin and subcutaneous tissue is infiltrated in the area of introduction that has been verified fluoroscopically;
2) a needle is then inserted through into the affected foramen; again fluoroscopic confirmation is noted;
3) the analyte wire with absorbent material is introduced through the needle;
4) a syringe containing approximately 1 cc of normal saline (NS) is attached to the needle;
5) NS is then injected soaking the cotton sponge;
6) the analyte wire is then removed;
7) the composition is then washed into a microcentrifuge tube containing a protease inhibitor cocktail solution and immediately placed on ice or dry ice or submerged in liquid nitrogen or any other method of rapid freezing;
8) the sample is then transferred to permanent storage at −20° C. or lower until analysis. Alternatively, at step seven the composition could then be immediately analyzed utilizing a centralized lab or point of care assay.

Method 5: Translaminar
1) the skin and subcutaneous tissue is infiltrated in the area of introduction;
2) a needle is then inserted utilizing an interlaminar approach to the epidural space. A “pop” through the ligamentum flavum will confirm appropriate position in the epidural space, alternatively, fluoroscopic confirmation may be utilized;
3) a syringe containing approximately 3-5 cc of normal saline (NS) is attached to the needle;
4) approximately 1 cc increments of NS are then injected into the epidural space with an attempt at aspiration with each volume of injectate, three to five seconds is allowed to elapse following each injection prior to reaspiration;
5) the aspirate is then placed in a microcentrifuge tube containing a protease inhibitor cocktail solution and immediately placed on ice or dry ice or submerged in liquid nitrogen or any other method of rapid freezing;
6) the sample is then transferred to permanent storage at −20° C. or lower until analysis.

Method 6: Disk Space (Lavage)
1) the skin and subcutaneous tissue is infiltrated in the area of introduction that has been verified fluoroscopically;
2) utilizing either a single or double needle technique, insertion into the disc space is accomplished under fluoroscopic guidance;
3) a syringe with approximately 1.5 cc of NS is attached to the needle and injected into the disc space;
4) after approximately 3 seconds the disc is re-aspirated for lavage fluid;
5) this may need to be repeated to obtain sufficient fluid (approximately ⅜ cc);
6) the aspirate is then washed into a microcentrifuge tube containing a protease inhibitor cocktail solution and immediately placed on ice or dry ice or submerbed into liquid nitrogen;
7) the sample is then transferred to permanent storage at −20° C. or lower until analysis.

Method 7: Disk Space (Absorbent Sponge)
1) the skin and subcutaneous tissue is infiltrated in the area of introduction that has been verified fluoroscopically;
2) utilizing either a single or double needle technique, insertion into the disk space is accomplished under fluoroscopic guidance;
3) the introducer guide wire is removed and the analyte wire with absorbent material is introduced;
4) a syringe containing approximately 1 cc of normal saline (NS) is attached to the introducer port;
5) NS is then injected soaking the cotton sponge.
6) the analyte wire is then removed
7) the composition is then washed into a microcentrifuge tube containing a protease inhibitor cocktail solution and immediately placed on ice or dry ice or submerbed in liquid nitrogen.
8) the sample is then transferred to permanent storage at −20° C. or lower until analysis. Alternatively, at step seven the composition could then be immediately analyzed utilizing a centralized lab or point of care assay.

Method 8: Facet Joint Space (Lavage)
1) the skin and subcutaneous tissue is infiltrated in the area of introduction that has been verified fluoroscopically;
2) utilizing either a single or double needle technique, insertion into the facet joint is accomplished under fluoroscopic guidance;
3) a syringe with approximately 1.5 cc of NS is attached to the needle and injected into the facet joint space;
4) after approximately 3 seconds the disc is re-aspirated for lavage fluid;
5) this may need to be repeated to obtain sufficient fluid (approximately ⅜ cc);
6) the aspirate is then washed into a microcentrifuge tube containing a protease inhibitor cocktail solution and immediately placed on ice or dry ice or submerbed into liquid nitrogen;
7) the sample is then transferred to permanent storage at −20° C. or lower until analysis.

Method 9: Facet Joint Space (Absorbent Sponge)
1) the skin and subcutaneous tissue is infiltrated in the area of introduction that has been verified fluoroscopically;
2) utilizing either a single or double needle technique, insertion into the facet joint is accomplished under fluoroscopic guidance;
3) the introducer guide wire is removed and the analyte wire with absorbent material is introduced;
4) a syringe containing approximately 1 cc of normal saline (NS) is attached to the introducer port;
5) NS is then injected soaking the cotton sponge.
6) the analyte wire is then removed
7) the composition is then washed into a microcentrifuge tube containing a protease inhibitor cocktail solution and immediately placed on ice or dry ice or submerbed in liquid nitrogen.
8) the sample is then transferred to permanent storage at −20° C. or lower until analysis. Alternatively, at step seven the composition could then be immediately analyzed utilizing a centralized lab or point of care assay.
   V. Diagnosis

The present methods can be used in the diagnosis, prognosis and treatment of radiculopathy, facet joint pain or discogenic pain.

In numerous embodiments of the present invention, the level and/or presence of a cytokine biomarker or its fragment, e.g., IFNγ or fragment thereof, polynucleotide or polypeptide will be detected in a biological sample, thereby detecting the presence or absence of radiculopathy or the presence or absence of discogenic pain, or the presence or absence of facet joint pain. In some embodiments, the biological sample will comprise a tissue sample from the spine, e.g. a fluid sample or lavasate of a disk or epidural space of a patient suspected of suffering from radiculopathy. In some embodiments, the biological sample will comprise a tissue sample from the spine, e.g., a fluid sample or lavasate of a disk space of a patient suspected of suffering from discogenic pain. In some embodiments, the biological sample will comprise a tissue sample from the spine, e.g., a fluid sample or lavasate of a facet joint of a patient suspected of suffering from facet pain.

In some embodiments, a biological sample determined to contain the cytokine biomarker, e.g., IFNγ or a fragment of IFNγ can be further analyzed to determine the levels of the cytokine biomarker, e.g., IFNγ or a fragment thereof. Determining the levels of a cytokine biomarker in a biological sample can aid in further characterizing the affected spine, e.g., the efficacy of certain treatments.

The amount of the cytokine biomarker polynucleotide or polypeptide, e.g., IFNγ or fragment thereof polynucleotide or polypeptide that will indicate the presence of radiculopathy or the presence of discogenic pain and classify the patient as candidate for treatment will depend on numerous factors, including the location of the affected disk along the spine, the age, sex, medical history, etc., of the patient, the cell type, the assay format, etc. In some embodiments, a level of cytokine biomarker, e.g., IFNγ or its fragment in a biological sample will not be quantified or directly compared with a control sample, but will rather be detected relative to a “diagnostic presence” of the cytokine biomaker wherein a “diagnostic presence” refers to an amount of the cytokine biomarker polynucleotide or polypeptide that indicates the presence or likelihood of radiculopathy or discogenic pain of the mammal from which the sample was taken. In some embodiments, a “diagnostic presence” will be detectable in a simple assay giving a positive or negative result, where a positive “detection” of a “diagnostic presence” of the cytokine biomarker polynucleotide or polypeptide, e.g., IFNγ polynucleotide or polypeptide indicates the presence of radiculopathy or discogenic pain or facet pain in the mammal.

The cytokine biomarker level need not be quantified for a “diagnostic presence” to be detected. Rather any method of determining whether the cytokine biomarker is present at levels higher than in a normal or control may be used. In addition, a “diagnostic presence” does not refer to any absolute quantity of a cytokine biomarker, but rather to an amount that, depending on the biological sample, assay conditions, medical condition of the patient, etc., is sufficient to distinguish the level in an affected patient from a normal or control patient.

Such methods can be practiced regardless of whether any cytokine biomarker polynucleotide or polypeptide, e.g., IFNγ (or fragment thereof) polynucleotide or polypeptide is normally present, or “expected” to be present, in a particular control sample. For example, the cytokine biomarker, e.g., IFNγ or its fragment may not be expressed in certain normal spine samples (such as, for example, in a disk space or epidural space lavasate) resulting in a complete absence of the cytokine biomarker in a control biological sample. For such biological samples, a “diagnostic presence” refers to any detectable amount of the cytokine biomarker, e.g., IFNγ or fragment thereof using any assay. In other instances, however, there may be a detectable level of the cytokine biomarker, e.g., IFNγ or a fragment thereof present in normal or control samples and a “diagnostic presence” represents a level that is higher than the normal level, preferably representing a “statistically significant” increase over the normal level. Often, a “diagnostic presence” of the cytokine biomarker polynucleotide, polypeptide, and/or protein activity in a biological sample will be at least about 1.5, 2, 5, 10,100, 200, 500, 1000 or more fold greater than a level expected in a sample taken from a normal patient or, for example, from the normal, unaffected or asymptomatic disk space of the same patient or in a group of individuals not suffering from pain of spinal origin

In some embodiments, the presence or level of the cytokine biomarker at a particular location along the spine is indicative of injury at that particular location. For example, if the cytokine biomarker protein is detected in the L5 disc lavasate, the patient has sustained injury at L5. The presence of the biomarker polypeptide can then be used to diagnose injury and administer treatment at a particular location irrespective of whether injury was detectable by other methods, e.g., an MRI. The patient will typically be treated by administration of a therapeutic agent to the site of injury, i.e., the site of presence of the cytokine biomarker. In other embodiments, when the patient is suffering from chronic pain, the presence of the cytokine biomarker, e.g., IFNγ, at any location along the spine is indicative of a patient to be selected for treatment, typically by systemic administration of a therapeutic agent.

The presence or level of polynucleotide, protein or polypeptide of a cytokine biomarker or fragments thereof can be used to designate a patient as candidate for treatment. The type of treatment, e.g., anti-inflammatory agent or surgery, can be then tailored to severity of the condition as determined by the presence or level of the cytokine biomarker, e.g., IFNγ or fragment thereof.

The present methods can also be used to assess the efficacy of a course of treatment. For example, in a patient with radiculopathy testing positive for a “diagnostic presence” of a cytokine biomarker indicative or radiculopathy, such as for example, IFNγ polynucleotide or polypeptide or fragment thereof, the efficacy of an anti-inflammatory treatment can be assessed by monitoring, over time, the levels of IFNγ or a fragment thereof. For example, a reduction in the cytokine biomarker's polynucleotide or polypeptide levels in a biological sample taken from a patient following a treatment, compared to a level in a sample taken from the mammal before, or earlier in, the treatment, indicates efficacious treatment.

VI. Treatment Methods

Once radiculopathy or discogenic pain or facet pain has been diagnosed, any number of methods known in the art for treating spinal related pain can be applied to treat the patient, e.g., Laminotomy, Laminectomy, discectomy, microdiscectomy, percutaneous discectomy, endoscopic discectomy, laser discectomy, foramenotomy, fusion, prolotherapy, other surgical decompressions, decompression with fusion with or without instrumentation.

A. Treatment Methods using Cytokine Biomarker Inhibitors

In some embodiments, an antibody that directly interacts with the cytokine biomarker detected in the assays of the invention can be used to treat radiculopathy or discogenic or facet pain. In some embodiments, if the patient is found to have a diagnostic presence of a cytokine biomarker in a disk space, injection of an antagonist for the particular cytokine biomarker can be done directly into the disk space. In other embodiments, if the patient suffering from spinal-related pain is found to have a diagnostic presence of a cytokine biomarker in the epidural space, an injection of the cytokine biomarker antagonist into the epidural space can alleviate radiculopathic pain. For example, an anti-IFNγ antibody may be used for therapeutic applications. For example, such an antibody may be conjugated to a protein that facilitates entry into the cell. In one case, the antibody enters the cell by endocytosis. In another embodiment, a nucleic acid encoding the antibody is administered to the individual or cell.

In one embodiment, the antibodies to the IFNγ protein are capable of reducing or eliminating a biological function of IFNγ as is described below. That is, the administration of anti-IFNγ antibodies (either polyclonal or monoclonal) to an injured disk can reduce or eliminate the pain associated with the injury.

A specific example of an anti-gamma interferon antibody is HuZAF (see, e.g. U.S. Pat. No. 6,329,511). HuZAF or an antibody that competes with HuZAF for binding to gamma interferon can be used with the treatment methods of the present invention.

Often, the antibodies to the IFNγ proteins for therapeutic applications are humanized antibodies (e.g., Xenerex Biosciences, Mederex, Inc., Abgenix, Inc., Protein Design Labs, Inc.) Humanized forms of non-human (e.g., murine) antibodies are chimeric molecules of immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

Human antibodies can also be produced using various techniques known in the art, including phage display libraries (Hoogenboom & Winter, J. Mol. Biol. 227:381 (1991); Marks et al., J. Mol. Biol. 222:581 (1991)). The techniques of Cole et al. and Boerner et al. are also available for the preparation of human monoclonal antibodies (Cole et al., Monoclonal Antibodies and Cancer Therapy, p. 77 (1985) and Boerner et al., J. Immunol. 147(1):86-95 (1991)). Similarly, human antibodies can be made by the introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg & Huszar, Intern. Rev. Immunol. 13:65-93 (1995).

In some embodiments, IFNγ antagonists for use in the invention are antibodies that bind IFNγ, e.g. fontolizumab. Such antibodies can be neutralizing antibodies that block IFNγ activity. Humanized antibodies that bind to IFNγ are described, e.g., in U.S. Pat. No. 6,329,511 and U.S. Pat. No. 7,183,390. In other embodiments, the antibody indirectly inhibits interferon gamma. IFNγ binding partner include, for example, interferon gamma receptor 1, interferon gamma receptor 2, TNFα and protein disulfide isomerase A3. Antibodies to IFNγ's binding partners can also be used to inhibit the activity of IFNγ and thus serve as antagonists of IFNγ. Other interferon antagonists useful for the treatment of interferon-related related diseases are described, for example, in U.S. Application 2003138404.

Other specific IFNγ antagonists include, e.g., soluble versions of IFNγ receptors (examples of known interferon gamma receptors are interferon gamma receptors 1 and 2), and soluble versions of other IFNγ binding partners. Viral proteins that are known to bind IFNγ can also be used as antagonists or inhibitors of IFNγ activity. For example, chimeric viroceptors which bind to various species of IFNγ, can be used as antagonists.

An inhibitor other than an anti-interferon gamma antibody can also assert its inhibitory effect indirectly by first interacting with a molecule in the same signaling pathway. Examples of such IFNγ inhibitors encompasses simple or complex organic or inorganic molecule, peptide, peptide mimetic, protein (e.g. antibody), liposome, small interfering RNA, or a polynucleotide (e.g. anti-sense) that can reduce the deleterious effect of IFNγ on radiculopathy or discogenic pain or facet pain.

Non-specific IFNγ inhibitors include matrix inhibitors; MMP inhibitors (i.e. such as: matrix Tetracyclines such as: metalloproteinase Prinomastat Doxycycline, inhibitors) (AG3340); Trovafloxacin, Batimastat; Lymecycline, Marimastat; Oxitetracycline, BB-3644; Tetracycline, KB-R7785; Minocycline, TIMP-1, and and synthetic TIMP-2, tetracycline adTIMP-1 derivatives, such (adenoviral delivery as CMT, i.e. of TIMP-1), and Chemically adTIMP-2 Modified (adenoviral delivery Tetracyclines; of TIMP-2); Quinolones (chinolones) such as: Norfloxacin, Levofloxacin, Enoxacin, Sparfloxacin, Temafloxacin, Moxifloxacin, Gatifloxacin, Gemifloxacin, Grepafloxacin, Trovafloxacin, Ofloxacin, Ciprofloxacin, Pefloxacin, Lomefloxacin, Temafloxacin, Rebamipide, and Nalidixic acid; Lazaroids; Pentoxifyllin derivates; Phosphodiesterase I, II, III, IV, and V-inhibitors; CC-1088, Ro 20-1724, rolipram, amrinone, pimobendan, vesnarinone, SB 207499.

In some embodiments of the present invention, the cytokine biomarker inhibitor or antagonist, e.g., an IFNγ inhibitor or antagonist administered to alleviate the symptoms of radiculopathic pain or discogenic or facet pain in administered in combination with another agent concurrently or consecutively with the proviso that the second agent is not an TNFα antagonist or inhibitor. In some embodiments of the present invention, the cytokine biomarker inhibitor or antagonist, e.g., an IFNγ inhibitor or antagonist administered to alleviate the symptoms of radiculopathic pain in administered in combination with another agent concurrently or consecutively with the proviso that the second agent is not an anti-TNFα antibody.

Nucleic Acid Inhibitors

Ribozymes, antisense RNA and/or small interfering RNA (siRNA) molecules can be screened for the ability to decrease the levels of a cytokine biomarker.

In some embodiments, siRNA molecules designed to target the IFNγ RNA can be used to inhibit the cytokine biomarker's activity. The phenomenon of RNA interference is described and discussed, e.g., in Bass, Nature 411:428-29 (2001); Elbahir et al., Nature 411:494-98 (2001); and Fire et al., Nature 391:806-11 (1998), where methods of making interfering RNA also are discussed. The siRNAs based upon the sequence of IFNγ are typically less than 100 base pairs, typically 30 base pairs or shorter, and are made by approaches known in the art. Exemplary siRNAs according to the invention could have up to 29 bps, 25 bps, 22 bps, 21 bps, 20 bps, 15 bps, 10 bps, 5 bps or any integer thereabout or there-between.

The siRNA can comprise two complementary molecules, or can be constructed such that a single transcript has both the sense and complementary antisense sequences from the target gene, e.g. a hairpin.

Methods for designing double stranded RNA to inhibit gene expression in a target cell are known (see, e.g., U.S. Pat. No. 6,506,559; Elbashir et al. Methods 26:199-213, 2002; Chalk et al., Biochem. Biophysy Res. Comm 319:264-274, 2004; Cui et al. Computer Method and Programs in Biomedicine 75:67-73, 2004, Wang et al., Bioinformatics 20:1818-1820, 2004). For example, design of siRNAs (including hairpins) typically follow known thermodynamic rules (see, e.g., Schwarz, et al., Cell 115:199-208, 2003; Reynolds et al., Nat Biotechnol. 22:326-30, 2004; Khvorova, et al., Cell 115:209-16, 2003). Many computer programs are available for selecting regions of IFNγ that are suitable target sites. These include programs available through commercial sources such as Ambion, Dharmacon, Promega, Invitrogen, Ziagen, and GenScript as well as noncommercial sources such as EMBOSS, The Wistar Institute, Whitehead Institute, and others.

For example, design can be based on the following considerations: typically shorter sequences, i.e., less than about 30 nucleotides are selected; the coding region of the mRNA is usually targeted; the search for an appropriate target sequence optionally begins 50-100 nucleotides downstream of the start codon, as untranslated region binding proteins and/or translation initiation complexes may interfere with the binding of the siRNP endonuclease complex. Some algorithms, e.g., based on the work of Elbashir et al., supra, search for a 23-nt sequence motif AA(N19)TT (N, any nucleotide) and select hits with approx. 50% G/C-content (30% to 70% has also worked in for them). If no suitable sequences are found, the search is extended using the motif NA(N21). The sequence of the sense siRNA corresponds to (N19)TT or N21 (position 3 to 23 of the 23-nt motif), respectively. In the latter case, the 3′ end of the sense siRNA is converted to TT.

Other algorithms preferentially select siRNAs corresponding to the target motif NAR(N17)YNN, where R is purine (A, G) and Y is pyrimidine (C, U). The respective 21-nt sense and antisense siRNAs therefore begin with a purine nucleotide and can also be expressed from pol III expression vectors without a change in targeting site; expression of RNAs from pol III promoters is only efficient when the first transcribed nucleotide is a purine.

Other nucleic acids, e.g., ribozymes, antisense, can also be designed based on known principles. For example, Sfold (see, e.g, Ding, et al., Nucleic Acids Res. 32 Web Server issue, W135-W141, Ding & Lawrence, Nucl. Acids Res. 31: 7280, 7301, 2003; and Ding & Lawrence Nucl. Acids Res. 20:1034-1046, 2001) provides programs relating to designing ribozymes and antisense, as well as siRNAs.

VII. Administration of Pharmaceutical Compositions

Therapeutic agents, e.g., IFNγ inhibitors can be administered to a patient for the treatment of radiculopathic, discogenic and facet pain. As described in detail below, the therapeutic agents are administered in any suitable manner, optionally with pharmaceutically acceptable carriers. Typically, the therapeutic agent is administered to the area of the spine where the cytokine biomarker is detected, e.g., when the cytokine biomarker is detected in the epidural space, the therapeutic agent is administered into the epidural space; when the cytokine biomarker is detected in the disk space, the therapeutic agent is administered into the disk space, when the cytokine biomarker is detected in the facet joint, the therapeutic agent is administered into the facet joint. In some embodiments, e.g., when the patient is suffering from chronic spinal pain, the therapeutic agent is administered systemically.

The therapeutic agents can be administered to a patient at therapeutically effective doses to prevent, treat, or control radiculopathic, discogenic and/or facet pain. The compounds are administered to a patient in an amount sufficient to elicit an effective protective or therapeutic response in the patient. An effective therapeutic response is a response that at least partially arrests or slows the symptoms or complications of the disease. An amount adequate to accomplish this is defined as “therapeutically effective dose.” The dose will be determined by the efficacy of a particular therapeutic agent, for example an anti-IFNγ antibody, employed and the condition of the subject, as well as the body weight of the patient to be treated. The size of the dose also will be determined by the existence, nature, and extent of any adverse effects that accompany the administration of a particular compound or agent in a particular subject.

Toxicity and therapeutic efficacy of such compounds can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Compounds that exhibit large therapeutic indices are preferred. While compounds that exhibit toxic side effects can be used, care should be taken to design a delivery system that targets such compounds to the site of affected tissue to minimize potential damage to normal cells and, thereby, reduce side effects.

The data obtained from cell culture assays and animal studies can be used to formulate a dosage range for use in humans. The dosage of such compounds lies preferably within a range of circulating concentrations that include the ED₅₀ with little or no toxicity. The dosage can vary within this range depending upon the dosage form employed and the route of administration. For any compound used in the methods of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC). In general, the dose equivalent of a modulator is from about 1 ng/kg to 10 mg/kg for a typical subject.

Pharmaceutical compositions for use in the present invention can be formulated by standard techniques using one or more physiologically acceptable carriers or excipients. The compounds and their physiologically acceptable salts and solvates can be formulated for administration by any suitable route, including via direct injection into the affected site.

Furthermore, the compounds can be formulated as a depot preparation. Such long-acting formulations can be administered by implantation (for example, subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device that can contain one or more unit dosage forms containing the active ingredient. The pack can, for example, comprise metal or plastic foil, for example, a blister pack. The pack or dispenser device can be accompanied by instructions for administration.

In some embodiments, the therapeutic agent is administered concurrently with sample extraction from the spine using the same device used for sample extraction or using a separate device.

VIII. Kits for Use in Diagnostic and/or Prognostic Applications

For use in diagnostic, research, and therapeutic applications suggested above, kits are also provided by the invention. In the diagnostic and research applications such kits may include any or all of the following: assay reagents, buffers, cytokine biomarker-specific nucleic acids or antibodies, hybridization probes and/or primers.

The kits of the present invention include selective binding partners for a cytokine biomarker, e.g., IFNγ or a fragment of IFNγ. In some embodiments, the kits of the present invention include the selective binding partners on a continuous solid surface.

In some kits, the selective binding partners are anti-IFNγ antibodies. In such kits, detection of the presence or level of IFNγ or a fragment thereof is by immunoassay. In some embodiments the kits of the present invention contain an anti-IFNγ antibody selected from the anti-INFγ antibody or antibodies in the Bio-Rad 17-plex panel available under catalog number 171A11171 (Bio-Rad, Hercules, Calif.).

In some embodiments, the kits include primers specific to amplifying a particular cytokine biomarker, e.g., IFNγ. Those of skill in the art can easily determine how to design primers specific for amplifying a particular cytokine biomarker based on its nucleotide sequence. Nucleotide detection methods can be used with kits comprising primers. In some embodiments, polymerase chain reactions are used to detect the cytokine biomarker(s). In some embodiments, the polymerase chain reaction is RT-PCR.

In some kits, a device to be utilized for the extraction of the biological sample is also included in the kit. In some embodiments, the extraction device, e.g., a syringe, a needle and a catheter, can directly extract the biological sample from the potentially affected disc or epidural space into a chamber containing the selective binding partners for the cytokine biomarker(s). In some instances, the kit, thus allows for immediate assessment of the presence and/or level of the cytokine biomarker, e.g., IFNγ or its fragment and, therefore, immediate diagnosis of a patient suffering from non-immune inflammatory acute joint injury. These types of kits are particularly suitable for use at the point of care. An example of a point of care diagnostic system is described in U.S. Pat. No. 6,267,722 which is incorporated herein by reference. Other devices whose design can be adapted for use with the kits of the present invention are described, for example, in U.S. Pat. Nos. 7,198,522 and 6,818,455.

In some embodiments, the kit may include a solution to be used for the extraction of the biological sample from the spine. This solution included in the kit can be, for example, a physiologic solution, e.g. saline. In some embodiments, the kit may include one or more therapeutic agents which can be administered through the same device as used for the extraction of the sample or through a different device.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

### Example 1

### Patient Selection and Disk Classification

The specific sample retrieval techniques, biochemical investigative methodologies, and instrumentation design derived from an ongoing observational study of continuous patients in a private practice setting. An investigation was carried out to determine a potential cytokine biomarker in a cohort of patients with radiculopathy. On a continuous basis, patients with lumbar radiculopathy in the presence of clinical and radiographic evidence of intervertebral disk pathology referred for lumbar epidural injection were offered study entry and informed consent. MRI was used to determine the presence of pathologic changes in the lumbar spine of each study participant. Each participant underwent lumbar epidural injection by a single orthopedic spine surgeon experienced in injection technique. A lavage of the epidural space was performed prior to the installation of corticosteroids for the treatment of the lumbar radiculopathy.

With the approval of a human investigational review board, patients ranging in age from 18 to 82 with lumbar radiculopathy with at least three weeks duration were included in the patient population of interest. Patients were broken into three groups. Group 1 involved asymptomatic volunteers with no history of spinal related pain. Group 2 involved patients with radiculopathy secondary to a compressive cause (herniated disk or spinal stenosis). Group 3 involved patients with primarily back pain with little or no neural compression. The patients were identified from a number of consecutive patients offered study enrollment after being referred to an orthopedic surgeon for evaluation of their radiculopathy. Patients with a history of corticosteroid medication within a three month period prior to the recommended epidural injection, those with chronic medical conditions (insulin-dependent diabetes mellitus, coronary artery disease requiring surgery or interventional cardiology) or systemic inflammatory disease were excluded from the study.

Demographic information was obtained including gender, age, insurance, work status, and VAS prior to the procedure. Additionally, subjects with no history of back pain, symptomatic herniated nucleus pulposus (NP) or lumbar radiculopathy were recruited to act as normal controls. All groups underwent a complete physical evaluation and a standard outcomes assessment questionnaire.

Each study participant underwent lumbar epidural injection by a single physician (GJS) as treatment for radiculopathy. The patient was placed prone on a flour-op table. The sacral hiatus was identified and marked using lateral fluoroscopy. A sterile prep and drape was then undertaken. After infiltrating the subcutaneous tissue with 1% Xylocaine with epinephrine, a 14 gauge epidural needle was utilized to enter the caudal epidural space. The stylet was removed and a neurologic catheter was then advanced using standard technique up to the posterior aspect of the disk. The position was confirmed fluoroscopically. An attempt was made to position the needle adjacent to the nerve roots felt to be responsible for the patient's symptoms on the basis of history, physical examination and imaging studies. After a negative aspiration for blood and cerebrospinal fluid, approximately 3-5 ml of sterile physiologic saline was infused into the epidural space and immediately withdrawn back into the same syringe. This lavasate, typically 1-2 ml, was then placed into 2 ml eppendorf tubes containing 130 μl of protease inhibitor cocktail tablets (Roche Diagnostics, Indianapolis, Ind.) dissolved in PBS (˜0.045 tablet/ml sample) and frozen at or below −20° C. If the study subject was either in group 2 or 3, then a cocktail of 0.25% marcaine and depo-medrol was administered into the epidural space prior to catheter removal.

The concentrations of 17 inflammatory cytokines (IFNγ, IL-2, IL-4, IL-5, IL-6, IL-7, IL-8, IL-10, IL-12, IL-13, IL-17, G-CSF, GM-CSF, TNF-α, IL-1β, MCP-1 and MIP-1β) were quantified in epidural lavage samples using the human 17-plex inflammatory cytokine panel and the Bio-Plex 200 System (Bio-Rad, Hercules, Calif.), following the manufacture's protocol with a 96-well plate format. This assay utilizes a sandwich style ELISA linked to polystyrene beads and fluorophores, and has been validated against standard ELISA's of human blood samples (de Jager, te Valthuis et al, 2003). Human nerve growth factor (hNGF) was also assayed in some samples.

The presence of the inflammatory cytokines was assessed by the immunoreactivity of a cytokine biomarker in the sample with the antibodies of the Bio-Plex 200 System (Bio-Rad, Hercules, Calif.). For example, what is termed INFγ in the following examples is a biomarker protein or peptide that is immunoreactive with the anti-IFNγ antibody in the Bio-Plex 200 System (Bio-Rad, Hercules, Calif.), and what is referred to “INFγ concentrations” is a measurement of the concentration of a biomarker protein or polypeptide as measured by the level of its reactivity with the anti-IFNγ antibody in the Bio-Plex 200 System (Bio-Rad, Hercules, Calif.).

Significantly higher IFNγ concentrations were measured in epidural lavage samples from patients with radiculopathic pain and concordant MRI-verified nerve root compression compared to those complaining primarily of low back pain or who have demonstrated signs of non-organic pain. In addition, a lavage sample demonstrating an IFNγ concentration of greater than 10 pg/ml was 95% predictive of experiencing pain relief from the epidural steroid injection. Furthermore, the lavasate from patients who underwent a follow-up epidural lavage after pain relief by epidural steroid injection had reduced or completely attenuated levels of IFNγ. These findings will be discussed in relation to potential pain mechanisms, epidural IFNγ as a diagnostic biomarker and therapeutic target and its potential use as an evidenced-based treatment guide

### Example 2

### Results of Analyses

TABLE 1
Control samples (Group 1) -samples from patients
who did not experience back pain

| Age | VAS | IFNγ | TNFα | IL-2 | IL-4 | IL-6 | IL-12 | MCP1 | MIP1β |
|---|---|---|---|---|---|---|---|---|---|
| | | 0 | 0.16 | 0 | 0 | 0 | 0 | 0 | 0 |
| 37 | 0 | 0 | 0.36 | 0 | 0 | 0.07 | 0 | 0 | 0 |
| 40 | 0 | 0 | 0.06 | 0 | 0 | 0.08 | 0 | 0 | 0 |
| 41 | 0 | 0 | 0.19 | 0 | 0 | 0.05 | 0 | 0 | 0 |

* cytokine levels are expressed in pg/ml

Patient samples were retrieved as described in Example 1 and analyzed as described. These data indicate that patients not experiencing back pain consistently test negative for the cytokine biomarker immunoreactive with the Bioplex 200 (Bio-Rad, Hercules, Calif.) anti-IFNγ antibody.

TABLE 2
Control samples (Group 2) - samples from patients who were
experiencing back pain not suspected to be radiculopathy

| | | | Resp. | | | | | | | |
|---|---|---|---|---|---|---|---|---|---|---|
| Age | VAS | Patient | steroid | IFN-gamma | TNF-a | IL-2 | IL-4 | IL-6 | IL-12 | MIP-1b |
| 36 | 7 | 25 | poor | 8.78 | 0 | 0 | 0 | 0 | 0 | 1.18 |
| 46 | 7 | 2 | poor | 0 | 0.71 | 1.18 | 0 | 0.83 | 0.45 | 0 |
| 44 | 8 | 4 | good | 0 | 0 | 0 | 0 | 0 | 0 | 0 |
| 33 | 7 | 7 | poor | 0 | 0 | 0 | 0 | 0 | 0 | 0 |
| 50 | 7 | 8 | poor | 0 | 1.09 | 0.48 | 0 | 2.12 | 0 | 0 |
| 70 | 6 | 17 | fair | 0 | 0 | 0 | 0 | 0 | 0 | 0 |
| 62 | 9 | 21 | fair | 5.8 | 0 | 13.79 | 2.71 | 4.19 | 4.41 | 9.94 |
| 20 | 8 | 23 | fair | 8 | 0 | 0 | 0 | 0 | 0 | 0.95 |
| 42 | 5 | 24 | fair | 6.95 | 0 | 0 | 1.43 | 0 | 0 | 1.1 |
| 46 | 8 | 43 | poor | 4.39 | 0 | 0 | 0 | 0 | 0 | 0.76 |
| 44 | 10 | 35 | poor | 5.67 | 0 | 0 | 0 | 0 | 0 | 1.22 |
| 53 | 8 | 41 | poor | 8.78 | 0 | 0 | 0 | 0 | 0 | 0.3 |
| 28 | 9 | 42 | poor | 4.61 | 0 | 0 | 0 | 0 | 0 | 1.1 |
| 33 | 10 | 44 | poor | 7.06 | 0 | 0 | 0 | 0 | 0 | 0.62 |
| 20 | 9 | 45 | fair | 4.29 | 0 | 0 | 0 | 0 | 0 | 0.36 |
| 43 | 8 | 47 | poor | 4.39 | 0 | 0 | 0 | 0 | 0 | 0.64 |
| 55 | 5 | 48 | fair-poor | 5.56 | 0 | 0 | 0 | 0 | 0.45 | 5.01 |
| 46 | 8 | 101 | poor | 0 | 0 | 0 | 0 | 0 | 0 | 0 |
| 58 | 8 | 31 | poor | 3.65 | 0 | 0 | 2.15 | 0 | 0 | 0.68 |
| 55 | 6 | 5 | good | 0 | 0.48 | 0 | 0 | 0.7 | 0 | 0 |
| 19 | 9 | 51 | poor | 8.89 | 0 | 0 | 0 | 0 | 0 | 0.54 |
| 55 | 9 | 92 | poor | 2.44 | 0 | 0 | 0 | 0 | 0 | 0 |
| 51 | 7 | 67 | fair | 22.92 | 0.05 | 0.54 | 0 | 0 | 0.1 | 1.36 |
| 54 | 6.5 | 239 | poor | 0 | 0.27 | 0 | 0 | 0.03 | 0.13 | 0.25 |

* cytokine levels are expressed in pg/ml

Patient samples were obtained as described in the previous examples. Steroid response (“Resp. steroid”) indicates response to steroids as assessed 3 months after treatment. Low levels or absence of a cytokine biomarker immunoreactive with the anti-IFNγ antibody of the Bioplex 200 assay (Bio-Rad, Hercules, Calif.) combined with an overall poor response to steroid therapy indicate that the pain this group of patients was experiencing was most likely not due to radiculopathy and confirm that pain associated with low levels of the cytokine biomaker immunoreactive with the anti-IFNγ antibody in the Bioplex 200 (Bio-Rad, Hercules, Calif.) is unlikely to be alleviated by steroid therapy.

Table 3 outlines the data for patients suspected of suffering from radiculopathic pain. As seen from the data, the initially elevated levels of the anti-IFNγ antibody immunoreactive protein correspond to a good to excellent response to steroid treatment.

TABLE 3
Radiculopathy patients (Group 3)

| | | | Response | | | | | | |
|---|---|---|---|---|---|---|---|---|---|
| Age | VAS | Patient | steroid | IFN-gamma | TNF-a | IL-2 | IL-4 | IL-6 | IL-12 |
| 41 | 3 | 1 | good | 5272 | 0.19 | 0 | 2.21 | 1.11 | 0 |
| 25 | 8.5 | 3 | good | 221.18 | 0.67 | 0.28 | 0 | 1.41 | 0.5 |
| 62 | 5.5 | 6 | good | 7363.49 | 0.22 | 1.22 | 2.9 | 0.17 | 0 |
| 32 | 5 | 9 | excellent | 6997.38 | 2.18 | 0 | 4.03 | 5.84 | 2.27 |
| 56 | 5.5 | 13 | excellent | 6993.66 | 0.67 | 0.89 | 3.33 | 1.44 | 0 |
| 40 | 5 | 15 | good | 211 | 0.2 | 0 | 0 | 0 | 0 |
| 41 | 8 | 16 | excellent | 714 | 1.34 | 0 | 0 | 3.81 | 0.41 |
| 48 | 9 | 18 | excellent | 1104.14 | 0 | 0 | 0 | 0 | 0 |
| 44 | 8 | 26 | excellent | 173 | 0 | 0 | 0 | 0 | 0 |
| 78 | 7 | 22 | excellent | 38.26 | 0 | 0 | 0 | 0 | 0 |
| 29 | 8 | 27 | excellent | 177 | 0 | 0 | 0 | 0 | 0 |
| 37 | 10 | 88 | Good | 48.66 | 0 | 0.26 | 0 | 1.59 | 0 |
| 39 | 6 | 122 | Excellent | 137.35 | 0 | 0.64 | 1.09 | 0 | 0.03 |
| 55 | 7 | 118 | good | 402.14 | 0 | 1.28 | 3.03 | 0.2 | 0.12 |
| 45 | 5 | 119 | Good | 59.44 | 0 | 0.95 | 0.42 | 0 | 0 |
| 29 | 8 | 89 | excellent | 152.02 | 0 | 0 | 0 | 0 | 0 |
| 57 | 6 | 59 | Excellent | 96.37 | 0.05 | 1.69 | 0 | 0 | 0.28 |
| 65 | 5 | 68 | excellent | 55.68 | 0.01 | 0.12 | 0 | 0 | 0.03 |
| 58 | 6 | 60 | Excellent | 148.16 | 0.11 | 2.89 | 0 | 0 | 0 |
| 47 | 5 | 62 | Excellent | 12.73 | 0 | 0.24 | 0 | 0 | 0 |
| 52 | 8 | 246 | Excellent | 1957.15 | 0.29 | 0 | OOR< | 0.02 | OOR< |
| 36 | 8 | 248 | Poor | 0 | 0.25 | 0.01 | 0 | 0.02 | 0 |

* cytokine levels are expressed in pg/ml

TABLE 4
Response of patients to steroid therapy
relative to initial IFNγ levels

| | IFNγ levels | IFNγ levels | Response to |
|---|---|---|---|
| Patient | pre-treatment | post-treatment | treatment |
| 1 | 173 | 0 | excellent |
| 2 | 1104.1 | 0 | excellent |
| 3 | 4.3 | 0 | fair |
| 4 | 6997.4 | 5.56 | excellent |
| 5 | 2.4 | 0 | poor |
| 6 | 6993.7 | 0 | excellent |
| 7 | 1957.2 | 0 | excellent |
| 8 | 0 | 0 | poor |
| 9 | 0 | 0 | poor |
| 10 | 0 | 0 | fair |
| 11 | 5013.8 | 0 | excellent |
| 12 | 15.3 | 0 | excellent |
| 13 | 177 | 152 | good |

* IFNγ levels are in pg/ml

Patient samples were obtained by epidural lavage. The patients' anti-IFNγ antibody reactive protein levels were initially assessed (pre-treatment) with concurrent steroid administration. Post-treatment levels of the anti-IFNγ antibody protein were assessed 3 months after the initial assessment/steroid administration. These data indicate that there is a direct correlation between initial levels of the polypeptide reactive with the Bioplex 200 (Bio-Rad, Hercules, Calif.) anti IFNγ antibody and response to steroid treatment.

The results shown above demonstrate that presence and levels of interferon-gamma, as assessed by the immunoreactivity of a biomarker peptide in the spine sample, can serve as a cytokine biomarker of radiculopathic pain.

### Example 4

### An Animal Model—Acute Electrophysiology

An acute electrophysiological animal model for disc herniation-induced sensitization in rats has been previously described by Cuellar et al. (2005), J. Neurophysiology. For the present study, with the additions of 1) pre-treating the dorsal root ganglion (DRG) with either saline or a neutralizing interferon gamma antibody in a randomized, blinded manner, prior to application of disk material (nucleus pulposus, NP; 2) performing epidural site lavages at baseline and at the 3 hour time-point after NP+ saline or NP+ interferon gamma antibody; 3) analyzing these lavasates with a rat Bio-Plex 9-plex cytokine assay, in a similar manner as that performed for our human study, all other aspects (animals, surgery, L5 dorsal root ganglion exposure, coccygeal disc exposure, recording and unit characterization, NP harvest and application, data analysis and statistics) were performed in a similar manner to that described by Cuellar et al. 2005, J. Neurophysiol

NP+ blocker group: Windup values did not increase significantly over time during stimulation. There was no change in absolute windup pre- vs. post-NP+ blocker during 0.3 Hz, 0.1 Hz or 1 Hz stimulation. There were no changes at any time point.

NP+ saline group: Windup increased from pre- to 180-min post-NP. There was a significant increase in the total number of spikes (area under the curve; AUC) during the 1-3.33 and 3.33-10 sec AD latency windows 180 min post-NP vs. pre-treatment (p<0.005 for both latency windows)

### Example 5

### Case Report 1

A 57 year-old woman was referred to an orthopedic surgeon for persistent, atypical left knee pain. Her family doctor had ordered an MRI scan of the left knee and had informed the patient that she had sustained a medial meniscus tear. She denied history of any trauma or other inciting event. Further questioning revealed a history of occasional buttock pain. The physical examination revealed that she had tenderness on the lateral side of the knee with questionable swelling. The examination was equivocal for either atypical radiculopathy (spinal origin) or internal derangement of the knee. The orthopedic surgeon reviewed the MRI scan of the left knee, noting chondromalacia and degenerative changes of the medial meniscus. The patient underwent lavage of the knee with 10 cc of normal saline and then injection with a preparation of 40mg of a corticosteroid preparation and 3 cc of 0.25% Marcaine. The lavasate was placed in a microfuge tube with protease inhibitor (Roche Diagnostics, Indianapolis, Ind.) dissolved in PBS (˜0.045 tablet/ml sample) and frozen at 20° C. Upon analysis for cytokine biomarkers, none were found. Follow-up two weeks later revealed no improvement in her knee pain.

Based on these findings, the orthopedic surgeon then prescribed the patient an MRI scan of the lumbar spine, which revealed a moderate sized L3/4 disk herniation with anterior impression on the thecal sac. At that point the orthopedic surgeon was faced with two imaging studies, each of which may have explained the source of the patient's presenting pain, however, it was difficult if not impossible for the physician to determine which of these entities should be treated—the spine or the knee (via a surgical approach, removing the meniscus tear). Therefore, the orthopedic surgeon performed an epidural lavage in an attempt to differentiate the source of the atypical radiculopathy as of spinal or knee origin. Approximately 4 ml of sterile physiologic saline was infused into the epidural space and immediately withdrawn back into the same syringe.

This lavasate was then placed into 2 ml eppendorf tubes containing 130 μl of protease inhibitor cocktail tablets dissolved in PBS and frozen at −20° C. The patient was then administered a cocktail of 0.25% marcaine and depo-medrol into the epidural space prior to catheter removal. Bio-Plex immunassay revealed the presence of interferon gamma or a similar protein reactive to an interferon gamma antibody, in the lavasate of the epidural space (Table 5 below).

The patient reported excellent relief of her symptoms of atypical radiculopathic pain two weeks following the epidural lavage and steroid injection. The patient remained pain-free three months later with no further complaints. This type of analysis can be utilized in medical cases that are confounding to the physician in order to target treatments and avoid unnecessary medical expenses and invasive procedures. This is an example of successful differentiation of the spine or knee as the origin of atypical radiculopathic pain.

TABLE 5
Analysis of cytokine biomarkers in 57 year old patient

| | | | SIDE OF | | | | | | | |
|---|---|---|---|---|---|---|---|---|---|---|
| ID | AGE | VAS | PAIN | MRI REPORT | IFN-gamma | TNF-a | IL-2 | IL-4 | IL-6 | IL-8 |
| knee sample | 57 | 5 | Left | Meniscus | 0 | 0.14 | 0 | 0 | 0 | 0.48 |
| | | | | tear | | | | | | |
| spine sample | 57 | 5 | Left | L3/4 HNP | 96.37 | 0.05 | 1.69 | 0 | 0 | 0 |

* cytokine levels are expressed in pg/ml

### Example 6

### Case Report 2—Sample from Facet Joint

A 17-year-old male who was a competitive pitcher described left-sided back pain that commenced after pitching in a game approximately 6 weeks before. The sensation was localized to the upper lumbar spine on the left-hand side. Tenderness was noted in the region. No other localizing findings were identified. An MRI scan was obtained which revealed a facet joint cyst at the level of the left L3/4 facet and a small disc bulge at L4/5.

It was unknown whether the subjective complaints of pain were emanating from the facet joints or from the disk bulge. Under fluoroscopic guidance, the facet joint cyst was aspirated with a 20 cc syringe followed by injection of ½ cc Celestone and ½ cc 0.25% Marcaine preparation. The aspirate of approximately 1 ml was then placed into a 2 ml eppendorf tube containing 130 μl of protease inhibitor cocktail tablets (Roche Diagnostics, Indianapolis, Ind.) dissolved in PBS (˜0.045 tablet/ml sample) and frozen at −20° C. temporarily until being shipped to Stanford University on dry ice where samples were alliquoted and stored at −80° C.

Bio-Plex immunoassay revealed the presence of interferon gamma or a similar protein reactive to an interferon gamma antibody, in the lavasate of the facet joint cyst. The patient noted relief following the injection and was able to pitch a short time thereafter. At the six-month follow-up visit, no complaints of lumbar spinal pain had been noted since the resolution of his symptoms. This type of analysis can be utilized in medical cases that are confounding to the physician in order to target treatments and avoid unnecessary medical expenses and invasive procedures.

### Example 7

### Patients with Discogenic Pain—Disk Lavage Samples

Patients who experienced back pain of greater than six months duration underwent disc lavage based upon abnormalities identified on an MRI scan as well as additional lavages of other disks found to be normal on MRI scan.

TABLE 6
Cytokine levels from patients with discogenic pain

| ID # | AGE | VAS | MRI** | IFNg | TNF-a | IL-2 | IL-4 | IL-6 | IL-8 |
|---|---|---|---|---|---|---|---|---|---|
| 2001-1 | 39 | 9 | 4 | 1235.47 | OOR< | 3.65 | 2.99 | OOR< | 0.37 |
| 2005-1 | 48 | 6 | 3 | 22.7 | OOR< | OOR< | OOR< | OOR< | OOR< |
| 2005-2 | | 6 | 3 | 9.22 | OOR< | OOR< | OOR< | 0.11 | OOR< |
| 2005-3 | | 6 | 2 | 10.09 | OOR< | OOR< | 5.58 | OOR< | OOR< |
| 2005-4 | | 5 | 2 | 4.71 | OOR< | 2.51 | OOR< | 0.04 | 0.68 |
| 2006-1 | 50 | 6 | 3 | 12.06 | OOR< | OOR< | OOR< | OOR< | OOR< |
| 2006-2 | | 6 | 3 | 16.89 | OOR< | OOR< | OOR< | OOR< | OOR< |
| 2007-1 | 48 | 7 | 3 | 98.67 | OOR< | OOR< | OOR< | OOR< | OOR< |
| 2007-2 | | 7 | 4 | 134.1 | OOR< | OOR< | OOR< | OOR< | OOR< |
| 2007-3 | | 7 | 3 | 94.83 | 0.03 | 2.56 | OOR< | OOR< | OOR< |
| 2007-4 | | 6 | 3 | 135.59 | OOR< | 0.92 | OOR< | OOR< | OOR< |
| 2008-1 | 53 | 6 | 4 | 110.86 | OOR< | 0.19 | OOR< | OOR< | 1.22 |
| 2008-2 | | 2 | 3 | 310.9 | 0.08 | 2.1 | OOR< | OOR< | OOR< |
| 2008-3 | | 1 | 3 | 2115.73 | 0.05 | 5.93 | OOR< | 0.98 | 0.24 |
| 2009-1 | 46 | 7 | 2 | 128.12 | OOR< | 0.64 | 0.36 | OOR< | 0.21 |
| 2009-2 | | 6 | 3 | 59.44 | OOR< | 0.95 | 0.42 | OOR< | 0.2 |
| 2009-3 | | 1 | 2 | 309 | OOR< | 1.9 | 1.25 | OOR< | 0.44 |
| 2009-4 | | 1 | 2 | 114.02 | OOR< | 0.4 | 2.21 | OOR< | 0.12 |
| 2010-1 | 54 | 4 | 4 | 27.95 | OOR< | OOR< | OOR< | OOR< | OOR< |
| 2010-2 | | 1 | 3 | 72.28 | OOR< | 2.02 | 0.59 | OOR< | OOR< |
| 3001-1 | 42 | 5 | 3 | 234.51 | OOR< | 0.43 | OOR< | OOR< | OOR< |
| 3001-2 | | 4 | 1 | 44.09 | OOR< | OOR< | OOR< | OOR< | OOR< |
| 3001-3 | | 4 | 1 | 524.18 | 0.01 | 2.83 | OOR< | OOR< | OOR< |
| 3002-1 | 37 | 4 | 5 | 69.41 | OOR< | OOR< | OOR< | OOR< | OOR< |
| 3002-2 | | 2 | 4 | 23.85 | 0.01 | 0.26 | 6.95 | OOR< | 0.27 |
| 3002-3 | | 6 | 2 | 74.57 | OOR< | OOR< | OOR< | OOR< | OOR< |
| 3002-4 | | 2 | 3 | 102.88 | 0.02 | 0.54 | OOR< | 0.37 | OOR< |
| 3003-1 | 56 | 4 | 5 | 800.55 | 0.03 | 4.6 | OOR< | OOR< | OOR< |
| 3003-2 | | 5 | 3 | 353.91 | 0.1 | 4.3 | OOR< | 1.74 | OOR< |
| 3005-1 | 52 | 2 | 2 | 429.74 | OOR< | 0.85 | 2.06 | OOR< | 0.14 |
| 3005-2 | | 3 | 3 | 110.13 | OOR< | 0.03 | OOR< | OOR< | 0.17 |
| 3005-3 | | 6 | 4 | 137.35 | OOR< | 0.64 | 1.09 | OOR< | 0.1 |
| 3005-4 | | 2 | 5 | 209.21 | OOR< | 0.99 | 0.37 | OOR< | 0.18 |
| 3005-5 | | 2 | 2 | 337.9 | OOR< | 1.7 | 1.11 | 0.03 | 0.31 |
| 3006-1 | 36 | 5 | 3 | 343.98 | OOR< | 1.84 | 0.77 | OOR< | 0.27 |
| 3006-2 | | 3 | 2 | 402.14 | OOR< | 1.28 | 3.03 | 0.2 | 0.18 |
| 3006-3 | | 1 | 1 | 72.71 | OOR< | 0.58 | 0.58 | OOR< | 0.1 |
| 3007-1 | 54 | 1 | 4 | 159.17 | OOR< | OOR< | 1.05 | OOR< | 0.25 |
| 3007-2 | | 6 | 5 | 152.86 | OOR< | OOR< | 0.88 | OOR< | 0.08 |
| 3007-3 | | 1 | 4 | 86.61 | 0.63 | 1.13 | 0.21 | OOR< | 0.29 |
| 3007-4 | | 1 | 4 | 352.8 | OOR< | 1.07 | 1.02 | OOR< | 0.14 |
| 3007-5 | | 1 | 2 | 263.13 | OOR< | 1.22 | 0.54 | 0.04 | 0.16 |
| 3010-1 | 43 | 5 | 4 | 45.25 | OOR< | OOR< | 0.34 | OOR< | 0.34 |
| 3010-2 | | 1 | 3 | 197.4 | OOR< | 0.39 | 1.23 | OOR< | 0.1 |
| 3010-3 | | 4 | 1 | 367.19 | OOR< | 0.35 | 1.21 | OOR< | 0.11 |
| 3011-1 | 43 | 3 | 2 | 97.83 | OOR< | 0.32 | 0.39 | OOR< | 0.19 |
| 3011-2 | | 4 | 2 | 158.58 | OOR< | 0.57 | 0.15 | OOR< | 0.29 |
| 3011-3 | | 1 | 1 | 100.95 | OOR< | OOR< | 0.22 | OOR< | 0.13 |
| 3013-1 | 20 | 4 | 3 | 282.5 | OOR< | 0.32 | 0.27 | OOR< | 0.55 |
| 3013-2 | | 6 | 2 | 71.63 | OOR< | 0.29 | 0.42 | OOR< | 0.1 |
| 3013-3 | | 1 | 1 | 92.19 | OOR< | 1.81 | 0.42 | OOR< | 0.17 |
| 3014-1 | 25 | 8 | 1 | 215.27 | OOR< | 1.83 | 0.5 | OOR< | OOR< |
| 3014-2 | | 8 | 2 | 2813.95 | 0.27 | 9.31 | 3.79 | 2.5 | 0.56 |
| 3014-3 | | 7 | 1 | 1221.43 | 0.09 | 4.58 | 3.62 | 1.12 | OOR< |
| 3015-1 | 21 | 3 | 1 | 104.63 | OOR< | 1.85 | OOR< | OOR< | OOR< |
| 3015-2 | | 5 | 2 | 180.86 | OOR< | 1.45 | OOR< | OOR< | OOR< |
| 3015-3 | | 7 | 2 | 228.28 | OOR< | 2.46 | OOR< | OOR< | OOR< |
| 3015-4 | | 3 | 1 | 464.56 | OOR< | 5.34 | 1.5 | OOR< | 0.07 |
| 3016-1 | 64 | 2 | 3 | 57.83 | OOR< | 2.16 | OOR< | OOR< | OOR< |
| 3016-2 | | 4 | 2 | 235.05 | OOR< | 0.48 | OOR< | OOR< | OOR< |
| 3017-1 | 50 | 5 | 2 | 40.41 | OOR< | 1.92 | OOR< | OOR< | OOR< |
| 3017-2 | | 6 | 3 | 338.17 | OOR< | 1.05 | OOR< | OOR< | OOR< |
| 3017-3 | | 4 | 3 | 316.47 | OOR< | OOR< | OOR< | OOR< | OOR< |
| 3018-1 | 43 | 5 | 2 | 87.67 | OOR< | 2.89 | OOR< | OOR< | OOR< |
| 3018-2 | | 4 | 2 | 71.8 | OOR< | OOR< | OOR< | OOR< | OOR< |
| 3018-3 | | 5 | 3 | 159.9 | OOR< | 0.43 | OOR< | OOR< | OOR< |

*cytokine levels are in pg/ml
**MRI grade classification according to Pfirrmann
OOR< indicates undetectable levels

TABLE 8
Control disk lavage samples from patients who did not exhibit pain

| | | IO | | | | | | | |
|---|---|---|---|---|---|---|---|---|---|
| ID # | AGE | VAS | MRI** | IFNg | TNF-a | IL-2 | IL-4 | IL-6 | IL-8 |
| BB | 16 | NA | 1 | OOR< | 0.03 | OOR< | OOR< | 0.14 | OOR< |
| 1 | | | | | | | | | |
| BB | | NA | 1 | 0.77 | 0.02 | OOR< | OOR< | 0.9 | OOR< |
| 2 | | | | | | | | | |
| BB | | NA | 1 | 0.15 | 0.03 | OOR< | OOR< | 0.39 | OOR< |
| 3 | | | | | | | | | |
| BB | | NA | 1 | 0.04 | 0.02 | OOR< | OOR< | 0.93 | OOR< |
| 4 | | | | | | | | | |
| BB | | NA | 1 | 1.18 | 0.02 | OOR< | 0.07 | 3.59 | OOR< |
| 5 | | | | | | | | | |
| BB | | NA | 1 | 0.26 | 0.02 | OOR< | OOR< | 0.19 | OOR< |
| 6 | | | | | | | | | |
| BB | | NA | 1 | OOR< | 0.02 | OOR< | OOR< | 0 | OOR< |
| 7 | | | | | | | | | |

*cytokine levels are in pg/ml
**MRI grade classification according to Pfirrmann
OOR< indicates undetectable levels

These data indicate that the presence of an anti-IFNγ immunoreactive cytokine biomarker polypeptide in disc lavage samples from multiple locations along the spine are indicative of chronic back pain and can be used as a selection criterion for a patient who can benefit from systemic therapy.

### Example 8

### Transforaminal Epidural Extractions—Patients with Radiculopathic Pain

The data in the following table (Table 9) were obtained from patients suffering from back pain using the transforaminal method of epidural sample extraction as described herein.

TABLE 9
Levels of cytokine biomarkers obtained using the transforaminal
method of epidural sample extraction

| | | | MRI | | | | | | |
|---|---|---|---|---|---|---|---|---|---|
| Patient | AGE | VAS | RESULTS | IFN-gamma | TNF-a | IL-2 | IL-4 | IL-6 | IL-8 |
| 1037 | 75 | 7 | stenosis | 7.92 | 0 | 0 | 1.24 | 0 | 0 |
| 1042 | 43 | 8 | HNP L4-5 | 30.57 | 0 | 2.09 | 0 | 0 | 0.32 |
| 210 | 41 | 9 | hnp L5-S1 | 2162.88 | 0.04 | 1.62 | 0 | 4.45 | 0 |
| 1047 | 38 | 9 | HNP L4-5 | 266.82 | 0 | 0.54 | 0 | 0 | 0 |
| 1199 | 61 | 8 | HNP L4-5 | 210.66 | 0 | 0 | 0 | 0 | 0 |
| 1054 | 55 | 6 | HNP L4-5, L5-S1 | 104.63 | 0 | 1.85 | 0 | 0 | 0 |
| 1198 | 51 | 6 | hnp L5-S1 | 2813.95 | 0.27 | 9.31 | 3.79 | 2.5 | 0.56 |
| 1074 | 49 | 7 | hnp L5-S1 | 1221.43 | 0.09 | 4.58 | 3.62 | 1.12 | 0 |
| 1072 | 56 | 5 | HNP L4-5 | 27.22 | 0 | 0.65 | 0.54 | 0 | 0 |
| 1075 | 56 | 6 | HNP L4-5 | 226.67 | 0 | 4.44 | 0 | 0 | 0 |
| 1073 | 56 | 6 | HNP L4-5 | 33.78 | 0 | 0 | 0 | 0 | 0 |
| 1078 | 62 | 7 | stenosis | 19.55 | 0 | 0 | 0 | 0 | 0 |

* cytokine levels are expressed in pg/ml

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications and accession numbers cited herein are hereby incorporated by reference.

What is claimed is:

1. A method of identifying a cytokine biomarker of radiculopathic pain or discogenic pain or facet pain, the method comprising:
a) providing a biological sample from the spine of a patient suspected to be suffering from radiculopathic pain or discogenic pain or facet pain, and
b) detecting the cytokine biomarker in the biological sample from the spine.

2. The method of claim 1, wherein the biological sample from the spine is a disc lavage sample or an epidural lavage sample or facet lavage or aspiration.

3. The method of claim 1, wherein the cytokine biomarker is interferon gamma or a fragment of IFN-γ.

4. The method of claim 1, wherein the biological sample from the spine is obtained using a technique selected from the group consisting of epidural space lavage, transforaminal epidural space lavage, translaminar epidural space lavage, epidural caudal sponge retrieval, transforaminal caudal sponge retrieval, disk space lavage, disk space caudal sponge retrieval or facet lavage or aspiration.

5. The method of claim 1, wherein detecting the cytokine biomarker comprises conducting an immunoassay.

6. The method of claim 1, wherein detecting the cytokine biomarker comprises detecting the level of nucleic acid in the biological sample.

7. The method of claim 6, wherein detecting the level of nucleic acid in the biological sample comprises detecting an amplification reaction.

8. A method of selecting a patient for treatment, wherein the patient is suspected of having radiculopathic pain or discogenic pain, the method comprising:
detecting a level of IFNγ or a level of a fragment of IFNγ in a biological sample from the spine, wherein the presence of the level of IFNγ or the level of a fragment of IFNγ is indicative of a patient to be selected for treatment.

9. The method of claim 8, wherein the biological sample from the spine is obtained using a technique selected from the group consisting of epidural space lavage, transforaminal epidural space lavage, translaminar epidural space lavage, epidural caudal sponge retrieval, transforaminal caudal sponge retrieval, disc space lavage, disc space caudal sponge retrieval or facet lavage or aspiration.

10. The method of claim 8, wherein detecting the presence of the level of IFNγ or a fragment of IFNγ comprises conducting an immunoassay.

11. The method of claim 8, wherein detecting the level of IFNγ or the level of the fragment of IFNγ comprises detecting the level of nucleic acid in the biological sample.

12. The method of claim 11, wherein detecting the level of nucleic acid in the biological sample comprises detecting an amplification reaction.

13. The method of claim 10, further comprising administering one or more therapeutic agents to the patient, wherein the one or more therapeutic agents are selected from the group consisting of an INFγ antagonist and a steroidal anti-inflammatory agent.

14. A method of reducing pain in a patient having radiculopathic pain or discogenic pain or facet pain, the method comprising administering an IFNγ antagonist to the patient in an amount sufficient to reduce a portion of radiculopathic pain or discogenic pain or facet pain.

15. The method of claim 14, wherein the IFNγ antagonist is administered into an epidural space or into a disk space of a spine of the patient.

16. The method of claim 14, wherein the IFNγ antagonist is an anti-IFNγ antibody.

17. The method of claim 16, wherein the anti-IFNγ antibody is a neutralizing antibody.

18. The method of claim 16, wherein the anti-IFNγ antibody is a monoclonal antibody.

19. The method of claim 18, wherein the monoclonal antibody is a humanized antibody.

20. The method of claim 14, further comprising administering at least one additional therapeutic agent.

21. The method of claim 20, wherein the additional therapeutic agent is an anti-inflammatory agent.

22. The method of claim 21, wherein the anti-inflammatory agent is a steroidal anti-inflammatory agent.

23. A kit for diagnosing back pain, the kit comprising:
a device for extracting a biological sample from a spine; and
an antibody panel having an antibody to IFNγ, wherein the antibody panel receives an extracted biological sample from the device and diagnoses spinal-related pain with an antibody to IFNγ and the extracted biological sample.

24. The kit of claim 23, wherein:
the antibody panel comprises a chamber, the antibody to IFNγ located in the chamber; and
the device for extracting the biological sample is directly communicating with the chamber of the antibody panel.

25. A method of obtaining a sample from a spine substantially at an area of pathology, comprising:
inserting a catheter into an epidural space of the spine;
passing the catheter near an area of pathology of the spine;
introducing a volume of fluid into the spine at the area of pathology;
extracting a sample from the spine substantially at the area of pathology.

26. The method of claim 25, further comprising identifying a cytokine biomarker in the sample.

27. The method of claim 26, wherein the cytokine biomarker is a fragment of interferon gamma.

28. The method of claim 26, further comprising inserting one or more therapeutic agents through the catheter into the spine at the area of pathology.

29. A method of obtaining fluid from a spine substantially at an area of pathology, comprising:
introducing a wire having an absorbent material;
moving the catheter near the area of pathology to locate the absorbent material over a lesion in the area of pathology;
extracting a sample from the spine substantially at the area of pathology.

30. The method of claim 29, further comprising identifying a cytokine biomarker in the sample.

31. The method of claim 30, wherein the cytokine biomarker is a fragment of interferon gamma.

32. The method of claim 31, further comprising inserting one or more therapeutic agents through the catheter into the spine at the area of pathology.

33. A method of obtaining fluid from a spine substantially at an area of pathology, comprising:
introducing volume of fluid through an affected foramen into an epidural space of the spine based on a confirmed area of pathology;
waiting a period of time after introducing said volume of fluid; and
extracting incrementally a sample from the spine substantially at the area of pathology.

34. The method of claim 33, further comprising identifying a cytokine biomarker in the sample.

35. The method of claim 34, wherein the cytokine biomarker is a fragment of interferon gamma.

36. The method of claim 35, further comprising inserting one or more therapeutic agents through the catheter into the spine at the area of pathology.

37. A method of obtaining a sample from a spine substantially at an area of pathology, comprising:
introducing through an affected foramen a wire having an absorbent material into an epidural space of the spine based on a confirmed area of pathology; and
extracting a sample from the spine substantially at the area of pathology.

38. The method of claim 37, further comprising identifying a cytokine biomarker in the sample.

39. The method of claim 38, wherein the cytokine biomarker is a fragment of interferon gamma.

40. The method of claim 39, further comprising inserting one or more therapeutic agents through the catheter into the spine at the area of pathology.

41. A method of obtaining fluid from a spine substantially at an area of pathology, comprising:
positioning a needle in the epidural space of a spine to pop through a ligamentum flavum of the spine;
introducing a volume of fluid into the spine substantially at the area of pathology;
waiting a period of time after introducing said volume of fluid; and
extracting a sample from the spine substantially at the area of pathology.

42. The method of claim 41, further comprising identifying a cytokine biomarker in the sample.

43. The method of claim 42, wherein the cytokine biomarker is a fragment of interferon gamma.

44. The method of claim 43, further comprising inserting one or more therapeutic agents through the catheter into the spine at the area of pathology.

45. A method of obtaining a sample from a spine, comprising:
introducing a needle into a disk space in the spine;
introducing a volume of fluid into the disc space through said needle;
waiting a period of time after introducing volume of fluid; and
extracting a sample from the disk space in the spine.

46. The method of claim 45, further comprising identifying a cytokine biomarker in the sample.

47. The method of claim 46, wherein the cytokine biomarker is a fragment of interferon gamma.

48. The method of claim 47, further comprising inserting one or more therapeutic agents through the catheter into the spine at the area of pathology.

49. The method of claim 45, wherein introducing the needle further comprises using a single or double needle technique to introduce the needle into the disk space.

50. A method of obtaining a sample from a spine, comprising:
introducing into the disk space a wire having an absorbent material;
removing the wire from the spine; and
extracting a sample from the disk space in the spine.

51. The method of claim 50, further comprising identifying a cytokine biomarker in the sample.

52. The method of claim 51, wherein the cytokine biomarker is a fragment of interferon gamma.

53. The method of claim 52, further comprising inserting one or more therapeutic agents through the catheter into the spine at the area of pathology.

54. The method of claim 50, wherein introducing a needle into a disk space of the spine further comprises using a single or double needle technique using fluoroscopic guidance.

55. The method of claim 50, wherein introducing the needle further comprises using a single or double needle technique to introduce the needle into the disk space.

56. A method for determining an approximate location of spinal injury in a spine, comprising:
extracting one or more samples from the spine;
testing the one or more samples to determine whether cytokine biomarkers are present; and
if cytokine biomarkers are present in the one or more samples, determining the approximate location of the spinal injury based on where the cytokine biomarkers in the one or more samples were extracted.

57. The method of claim 56, wherein extracting one or more samples comprises extracting one or more samples from a plurality of extracting locations along the spine.

58. The method of claim 57, wherein determining the approximate location of the spinal injury based on the cytokine biomarkers in the one or more samples comprises:
determining a highest level sample with the highest level of cytokine biomarkers of the one or more samples by comparing levels of cytokine biomarkers in the one or more samples; and
determining the approximate location of the spinal injury based on the extracting location associated with the highest level sample.

59. The method of claim 56, wherein determining the approximate location of the spinal injury based on the cytokine biomarkers in the one or more samples comprises determining the approximate location of the spinal injury based on comparing one or more levels of cytokine biomarkers in the one or more samples.

60. The method of claim 56, further comprising inserting one or more therapeutic agents into the spine substantially at an area of pathology.

61. A method of obtaining a sample from a spine substantially at an area of pathology, comprising:
introducing a volume of fluid at a facet joint of the spine; and
extracting a sample from the spine substantially at the area of pathology.

62. The method of claim 61, further comprising identifying a cytokine biomarker in the sample.

63. The method of claim 62, wherein the cytokine biomarker is a fragment of interferon gamma.

64. The method of claim 63, further comprising inserting one or more therapeutic agents into the spine substantially at the area of pathology.

65. A method of obtaining a sample from a spine, comprising:
introducing into a facet joint in the spine a wire having an absorbent material; and
removing the wire from the spine to obtain a sample.

66. The method of claim 65, further comprising identifying a cytokine biomarker in the sample.

67. The method of claim 66, wherein the cytokine biomarker is a fragment of interferon gamma.

68. The method of claim 67, further comprising inserting one or more therapeutic agents into the spine substantially at an area of pathology.

69. A method of reducing pain in a patient having radiculopathic pain or discogenic pain or facet pain, the method comprising introducing a volume of fluid to a spinal anatomy, the volume of fluid sufficient to dilute one or more biological pain factors of the patient by an amount sufficient to reduce a portion of radiculopathic pain or discogenic pain or facet pain.

70. The method of claim 69, wherein the volume of fluid is introduced at intervals.

71. The method of claim 69, wherein introducing the volume of fluid comprises internally or externally supplying the volume of fluid to the patient.

72. The method of claim 69, wherein the volume of fluid is introduced at intervals independent of patient activity.

73. The method of claim 69, wherein introducing the volume of fluid is performed at patient discretion.

74. A method of reducing pain in a patient having joint pain, the method comprising introducing a volume of fluid to a specific anatomy, the volume of fluid sufficient to dilute one or more biological pain factors to the patient in an amount sufficient to reduce a portion of the joint pain.

75. The method of claim 74, wherein the volume of fluid is introduced at intervals.

76. The method of claim 74, wherein introducing the volume of fluid comprises internally or externally supplying the volume of fluid to the patient.

77. The method of claim 74, wherein the volume of fluid is introduced at intervals independent of patient activity.

78. The method of claim 74, wherein introducing the volume of fluid is performed at patient discretion.

79. The method of claim 69, wherein the volume of fluid is introduced based upon patient activity or physiological indicators.

80. The method of claim 74, wherein the volume of fluid is introduced based upon patient activity or physiological indicators.
